(19) United States
(12) Patent Application Publication
(10) Pub. No.: US 2021/0186888 A1
(43) Pub. Date: Jun. 24, 2021

(54) NANOPARTICLE COMPOSITIONS FOR CANCER TREATMENT

(71) Applicant: Wake Forest University Health Sciences, Winston-Salem, NC (US)

(72) Inventors: Ravi SINGH, Gastonia, NC (US); Jessica SWANNER, Winston-Salem, NC (US); Cale FAHRENHOLTZ, Winston-Salem, NC (US)

(21) Appl. No.: 16/622,128

(22) PCT Filed: Jun. 12, 2018
(86) PCT No.: PCT/US2018/037053

§ 371 Date: Dec. 12, 2019

Related U.S. Application Data
(60) Provisional application No. 62/518,269, filed on Jun. 12, 2017.

(51) Int. Cl. A61K 9/51; A61K 31/4436; A61K 33/38; A61K 45/06; G01N 33/574

(52) U.S. Cl. CPC A61K 9/5115; A61K 31/4436; G01N 33/57484; A61K 45/06; A61K 9/5138; A61K 33/38

(57) ABSTRACT

In one aspect, methods of treating cancer are disclosed. A method described herein, in some embodiments, comprises quantifying ZEB1 expression in a cancerous cell population and administering silver nanoparticles to the cancerous population if the quantified ZEB1 expression meets or exceeds a ZEB1 expression threshold. In some embodiments, the quantified ZEB1 expression is compared to a ZEB1 expression threshold above which a cancerous cell population response to silver nanoparticles.

### CROSS-REFERENCE TO RELATED APPLICATIONS

This application claims priority pursuant to 35 U.S.C § 119(e) to U.S. Provisional Patent Application Ser. No. 62/518,269, filed on Jun. 12, 2017, which is hereby incorporated by reference in its entirety.

### STATEMENT OF GOVERNMENT RIGHTS

This invention was made with government support under Grant Nos. T32CA079448, ROOCA154006 and CCSG P30CA012197 awarded by the National Cancer Institute (NCI). The government has certain rights in the invention.

### FIELD

The present invention relates to nanoparticle compositions for cancer treatment and, in particular, to nanoparticle compositions for selective treatment of cancerous cell populations exhibiting elevated ZEB1 expression.

### BACKGROUND

Triple negative breast cancer (TNBC) is characterized by the lack or decreased expression of the estrogen, progesterone, and human epidermal growth factor (HER2) receptors. As a consequence, TNBC patients do not benefit from modern receptor-targeted therapies and their prognosis is significantly worse than non-TNBC patients. Moreover, TNBC patients have a significantly higher risk of recurrence than patients with other types of breast cancer. Other than conventional cytotoxic chemotherapy, few systemic treatments are available and disease heterogeneity has limited the development of molecularly targeted therapeutics. Based upon advances in molecular profiling, TNBC can be further divided into Claudin-low breast cancer (CLBC) which makes up approximately 25-39% of TNBC cases and basal-like breast cancer which makes up the remaining TNBC cases. Claudin-low tumors and cell lines are enriched for markers of the mesenchymal metabolomic signature which includes cancer stem cell/tumor initiating cells (CSC/TIC) including CD44+/CD24−/low. CSC/TICs are radiation and chemotherapy resistant, and may be responsible for tumor relapse. The zinc-finger-E-box binding homeobox factor (ZEB1) plays a direct role in the induction of the epithelial-mesenchymal transition (EMT) via transcriptional repression of E-cadherin by direct binding to E-box sequences within the E-cadherin promotor and upregulate mesenchymal markers such as N-cadherin and vimentin. ZEB1 has been shown to repress the epithelial splicing regulatory protein 1 (ESRP1) which is necessary for the maintenance of the epithelial phenotype. Early evidence suggests CLBC tumors may be more resistant to neoadjuvant anthracycline/taxane-based chemotherapy compared to basal-like tumors. More effective, less toxic treatments for TNBC, with emphasis on the CLBC molecular subtype, are needed. Additionally, biomarkers to identify patients that will benefit the most from particular therapies are important to prevent resistance. As breast cancer comprises a group of heterogenous diseases, it becomes increasingly important to identify cancer cell specific vulnerabilities that can be exploited as therapeutic targets.

### SUMMARY

Silver nanoparticles (AgNPs) possess a desirable combination of selective cytotoxicity and radiation dose enhancement effects for treatment of TNBC cells at doses that are non-toxic to non-cancerous breast and other cells. AgNPs are pleotropic stressors and it is important to understand the mechanisms underlying AgNP toxicity and selectivity in order to define their application range and to optimize their therapeutic efficacy. Even at non-lethal doses, exposure to AgNPs can lead to oxidative stress or DNA damage. In addition, AgNP exposure also can cause endoplasmic reticulum (ER) stress which initiates the unfolded protein response (UPR). The UPR is an important cellular self-protection mechanism; yet, chronic activation of UPR due to stress that exceeds the capacity for cellular self-protection leads to apoptosis and cell death. ER stress is emerging as an Achilles heel for some cancers and exploiting this vulnerability may offer a novel route to selective cancer therapy.

A clear demonstration of selectivity for cancer cells and harmlessness for normal tissue homeostasis is critical for the translation of AgNPs for treatment of CLBC. Furthermore, little is known about the sublethal effects of AgNPs on the normal breast epithelium. Small changes in NP characteristics have the potential to dramatically change their toxicity profile. It is therefore necessary to clearly establish the relationships between structural and physicochemical AgNP characteristics and biological effects. Several embodiments described herein address these issues. Colloidal characterization of various sizes of AgNPs was performed, and the safety and CLBC-selectivity of these particles in monolayer cell culture, 3D mammary gland, or tumor organoid models was evaluated. The impact of AgNPs on the induction of the UPR, antioxidant levels, ROS levels, DNA damage, and apoptosis in CLBC and non-cancerous breast epithelial cells in vitro was quantified. Furthermore, an in vivo study to determine the efficacy of intravenously administered AgNPs for treatment of CLBC xenografts was performed. This is the first time a CLBC-specific treatment has been discovered. Biomarkers that can further pinpoint cancers, beyond CLBC, that may benefit from AgNP treatment were identified.

Silver nanoparticles (AgNPs) are cytotoxic to TNBC cells at doses that are non-cytotoxic to non-cancerous breast epithelial cells or to breast cancer cells corresponding to other molecular subtypes. Embodiments disclosed herein demonstrate that AgNPs are most effective against the CLBC subtype, and these cytotoxic properties are independent of particle size, shape or capping agent. The CLBC-specific cytotoxicity of AgNPs is not achieved using ionic silver and is therefore one of the first examples of a “new to nano” cytotoxic property. Mechanistically, the AgNPs deplete cellular antioxidants, induce the unfolded protein stress response (UPR), and eventually result in apoptotic cell death in CLBC cells without causing similar damage or cell death in non-cancerous breast epithelial cells. Furthermore, the AgNPs do not disrupt the normal architecture of breast acini in 3D cell culture, nor cause DNA damage or induce apoptosis in these structures. In contrast, the same doses of AgNPs cause extensive DNA damage and apoptosis in CLBC tumor nodules produced in 3D culture. Systemic administration of AgNPs is safe and effective for treatment of CLBC xenografts in mice. Basal levels of reactive oxygen species (ROS) correlate with sensitivity to AgNPs and induce degradation of AgNPs in CLBC. Furthermore, mesenchymal cancers, beyond CLBC, which can be defined by the biomarker pair disclosed herein, ZEB1^{High}/ESRP1^{Low}, are sensitive to AgNP treatment. The disclosure provides evidence that a therapeutic window exists for the safe use of AgNPs, which may strongly benefit the CLBC patient population for which prognoses are poor, as well as other mesenchymal cancers with equivalently poor prognoses.

In one aspect, methods of treating cancer are described herein. In some embodiments, a method comprises quantifying ZEB1 expression in a cancerous cell population, comparing the quantified ZEB1 expression with a ZEB1 expression threshold above which the cancerous cell population responds to silver nanoparticles, and administering silver nanoparticles to the cancerous cell population if the quantified ZEB1 expression meets or exceeds the ZEB1 expression threshold. In some embodiments, the cancerous cell population comprises triple-negative breast cancer cells. The triple-negative breast cancer cells can comprise claudin-low subtypes. The cancerous cell population, in other embodiments comprises, at least one of lung cancer cells, colorectal cancer cells, ovarian cancer cells and prostate cancer cells. In other cases, the cancerous cell population can comprise claudin-low subtypes.

Silver nanoparticles can be administered in any desired concentration consistent with the objectives of the present invention. In some embodiments, for example, the silver nanoparticles are administered at a concentration of silver of 1 μg/ml to 100 μg/ml or 5 μg/ml to 50 μg/ml. In some cases, the silver nanoparticles have an average size of 5 nm to 50 nm or 5 nm to 30 nm.

The silver nanoparticles can comprise a polymeric coating or a silica coating. In other embodiments, the silver nanoparticles are in the ground state.

In other embodiments, a method of treating cancer further comprises quantifying ESRP1 and/or CDH1 expression in the cancerous cell population. In some cases, the ESRP1 and/or CDH1 expression is less than ZEB1 expression.

In still further embodiments, the silver nanoparticles selectively kill the cancerous cell population. The silver nanoparticles, in some cases, are administered intravenously.

The cancerous cell population, in some embodiments, comprises elevated reactive oxygen species that trigger pH-dependent ionization of the silver nanoparticles.

In some embodiments, the silver nanoparticles reduce or inhibit the activation of heat shock factor 1 (HSF1).

Additionally, a method of treating cancer can further comprise administering one or more heat shock inhibitors. The one or more heat shock inhibitors can inhibit one or more heat shock proteins and/or HSF1. The one or more heat shock inhibitors, in some cases, synergizes with the silver nanoparticles.

In some cases, a method further comprises quantifying HSF1 activation, comparing the quantified HSF1 activation with a HSF1 activation threshold above which the cancerous cell population responds to an HSF1 inhibitor, and administering an HSF1 inhibitor to the cancerous cell population if the quantified HSF1 activation meets or exceeds the HSF1 activation threshold.

In another aspect, methods of determining response of a cancerous cell population to silver nanoparticles are described herein. In some embodiments, a method comprises quantifying ZEB1 expression in the cancerous cell population, and comparing the quantified ZEB1 expression with a ZEB1 expression threshold above which the cancerous cell population responds to the silver nanoparticles.

In some embodiments, a method further comprises quantifying endothelial splicing regulatory protein 1 (ESRP1) expression in the cancerous cell population, and comparing the quantified ESRP1 expression with an ESRP1 expression threshold below which the cancerous cell population responds to the silver nanoparticles.

In some embodiments, a method further comprises quantifying E-cadherin (CDH1) expression in the cancerous cell population, and comparing the quantified CDH1 expression with a CDH1 expression threshold below which the cancerous cell population responds to the silver nanoparticles.

In other embodiments, a method further comprises quantifying HSF1 activation in the cancerous cell population, and comparing the quantified HSF1 activation with a HSF1 activation threshold below which the cancerous cell population responds to the silver nanoparticles.

These and other embodiments are described in the detailed description below.

### BRIEF DESCRIPTION OF THE FIGURES

FIG. 1A is a transmission electron microscopic image AgNPs having a diameter of 5 nm, 25, nm, 50 nm, or 75 nm.

FIG. 1B is a hydrodynamic analysis of AgNPs having a diameter of 5 nm, 25, nm, 50 nm, or 75 nm.

FIG. 1C is a hydrodynamic light scattering analysis of AgNPs having a diameter of 25 nm in water, saline, or cell culture media.

FIG. 1D is an MTT assay of breast cancer cells and non-tumorigenic breast cells treated with AgNPs having a diameter of 5 nm, 25, nm, 50 nm, or 75 nm.

FIG. 1E is an MTT assay of breast cancer cells and non-tumorigenic breast cells treated with AgNPs having a diameter of 25 nm or silver nitrate.

FIG. 1F is an MTT assay of breast cancer cells and non-tumorigenic breast cells treated with gold nanoparticles.

FIG. 1G is an MTT assay of breast cancer cells and non-tumorigenic breast cells treated with silica-shelled, triangular nanoparticles.

FIG. 2A is an MTT assay of breast cancer cells and non-tumorigenic breast cells treated with AgNPs for 48 h.

FIG. 2B is an MTT assay of breast cancer cells and non-tumorigenic breast cells treated with AgNPs for 72 h.

FIG. 3A is a tumor growth plot from tumor bearing mice treated with AgNPs.

FIG. 3B is a tumor weight plot from tumor bearing mice treated with AgNPs.

FIG. 3C is a Kaplan-Meier plot of tumor bearing mice treated with AgNPs.

FIG. 3D is a photograph of tumor bearing mice treated with AgNPs.

FIG. 3E a bar graph of residual silver quantified in tumors resected from tumor bearing mice treated with AgNPs.

FIG. 3F is a bar graph of residual silver quantified in organs of tumor bearing mice treated with AgNPs.

FIG. 3G is a line graph of residual silver quantified in blood of tumor bearing mice treated with AgNPs.

FIG. 3H is a bar graph of residual silver quantified in urine of tumor bearing mice treated with AgNPs.

FIG. 4A is a bar graph of silver quantified in of breast cancer cells and non-tumorigenic breast cells treated with AgNPs.

FIG. 4B is a transmission electron microscopy image of non-tumorigenic breast cells treated with AgNPs.

FIG. 4C is a transmission electron microscopy image of breast cancer cells treated with AgNPs.

FIG. 5A is a line graph of plasmon resonance absorption of AgNP suspensions treated with H₂O₂ at pH 4 or pH 7.

FIG. 5B is a fluorescence microscopy image of breast cancer cells and non-tumorigenic breast cells treated with NAC or AgNPs.

FIG. 6A is a Redox Assay of breast cancer cells and non-tumorigenic breast cells treated with AgNPs.

FIG. 6B is a Redox Assay of breast cancer cells and non-tumorigenic breast cells treated with AgNPs.

FIG. 6C is an MTT assay of breast cancer cells treated with AgNPs.

FIG. 6D is a Western Blot analysis of breast cancer cells and non-tumorigenic breast cells.

FIG. 7A is a Western Blot analysis of breast cancer cells and non-tumorigenic breast cells treated with AgNPs.

FIG. 7B is a flow cytometry analysis of breast cancer cells and non-tumorigenic breast cells treated with AgNPs.

FIG. 7C a cell cycle analysis of breast cancer cells and non-tumorigenic breast cells treated with AgNPs.

FIG. 8A is a confocal microscopy image of S1 acini treated with AgNPs.

FIG. 8B is bar graph quantifying ZO-1 in S1 acini treated with AgNPs.

FIG. 8C is a confocal microscopy image of S1 acini treated with AgNPs.

FIG. 8D is a confocal microscopy image of S1 acini treated with AgNPs.

FIG. 8E is a confocal microscopy image of S1 acini treated with AgNPs.

FIG. 8F is bar graph quantifying apoptotic cells in S1 acini treated with AgNPs.

FIG. 8G is a confocal microscopy image of S1 acini treated with AgNPs or ionizing radiation.

FIG. 8H is bar graph quantifying DNA repair in S1 acini treated with AgNPs or ionizing radiation.

FIG. 8I is bar graph quantifying DNA damage in S1 acini treated with AgNPs or ionizing radiation.

FIG. 8J is bar graph quantifying apoptotic cells in S1 acini treated with AgNPs or ionizing radiation.

FIG. 9A is a scatter plot quantifying ZEB1 and ESRP1 expression in breast cancer cells and an MTT Assay of breast cancer cells treated with AgNPs.

FIG. 9B is a Western Blot analysis of breast cancer cells and non-tumorigenic breast cells.

FIG. 9C is light microscopy image of non-tumorigenic breast cells treated with TGF-β.

FIG. 9D is a Western Blot analysis of non-tumorigenic breast cells treated with TGF-β.

FIG. 9E is a fluorescence microscopy image of non-tumorigenic breast cells treated with TGF-β.

FIG. 9F is an MTT Assay of non-tumorigenic breast cells treated with TGF-β and AgNPs.

FIG. 9G is a Western Blot analysis of breast cancer cells treated with shRNA.

FIG. 9H is a Western Blot analysis of breast cancer cells treated with shRNA and AgNPs.

FIG. 9I is a fluorescence microscopy image of breast cancer cells treated with shRNA and AgNPs.

FIG. 10A is an MTT Assay of ovarian cancer cells treated with AgNPs for 24 h.

FIG. 10B is an MTT Assay of ovarian cancer cells treated with AgNPs for 72 h.

FIG. 10C is a fluorescence microscopy image of ovarian cancer cells.

FIG. 10D is a scatter plot quantifying ZEB1 and ESRP1 expression in ovarian cancer cells.

FIG. 10E is a Western Blot analysis of ovarian cancer cells.

FIG. 11A is a scatter plot quantifying ZEB1 and ESRP1 expression in lung cancer cells.

FIG. 11B is an MTT Assay of lung cancer cells treated with AgNPs for 72 h.

FIG. 11C is a scatter plot quantifying ZEB1 and ESRP1 expression in colorectal cancer cells.

FIG. 11D is an MTT Assay of colorectal cancer cells treated with AgNPs for 72 h.

FIG. 11E is a scatter plot quantifying ZEB1 and ESRP1 expression in prostate cancer cells.

FIG. 11F is an MTT Assay of prostate cancer cells treated with AgNPs for 72 h.

FIG. 12 is a cartoon of AgNP treatment in ZEB1/ESRP1 expressing cells.

FIG. 13 is a scatter plot of AgNP sensitivity relative to ZEB1 expression in cells.

FIG. 14 is a scatter plot of AgNP tolerance relative to ESRP1 expression in cells.

FIG. 15 is a scatter plot of AgNP sensitivity relative to CDH1 expression in cells.

FIG. 16 is a Western Blot analysis of breast cancer cells and normal cells treated with AgNPs.

FIG. 17A is an MTT Assay of BT549 Claudin Low Breast Cancer cells treated with AgNPs and/or an HSF1 inhibitor.

FIG. 17B is a scatterplot quantifying the dose reduction index of BT549 Claudin Low Breast Cancer cells treated with AgNPs or an HSF1 inhibitor.

FIG. 17C is a scatterplot quantifying the combination index of BT549 Claudin Low Breast Cancer cells treated with AgNPs and an HSF1 inhibitor.

FIG. 18A is an MTT Assay of BT20 basal breast cancer cells treated with AgNPs and/or an HSF1 inhibitor.

FIG. 18B is a scatterplot quantifying the dose reduction index of BT20 basal breast cancer cells treated with AgNPs or an HSF1 inhibitor.

FIG. 18C is a scatterplot quantifying the combination index BT20 basal breast cancer cells treated with AgNPs and an HSF1 inhibitor.

FIG. 19A is an MTT Assay of BT549 Claudin Low Breast Cancer cells treated with AgNPs and/or an HSP90 inhibitor.

FIG. 19B is a scatterplot quantifying the dose reduction index of BT549 Claudin Low Breast Cancer cells treated with AgNPs or an HSP90 inhibitor.

FIG. 19C is a scatterplot quantifying the combination index of BT549 Claudin Low Breast Cancer cells treated with AgNPs and an HSP90 inhibitor.

### DETAILED DESCRIPTION

Embodiments described herein can be understood more readily by reference to the following detailed description and examples. Methods, devices, and features described herein, however, are not limited to the specific embodiments presented in the detailed description and examples. It should be recognized that these embodiments are merely illustrative of the principles of the present disclosure. Numerous modifications and adaptations will be readily apparent to those of skill in the art without departing from the spirit and scope of the disclosure.

In addition, all ranges disclosed herein are to be understood to encompass any and all subranges subsumed therein. For example, a stated range of “1.0 to 10.0” should be considered to include any and all subranges beginning with a minimum value of 1.0 and ending with a maximum value of 10.0 or less, e.g. 1.0 to 5.3, or 4.7 to 10.0, or 3.6 to 7.9.

All ranges disclosed herein are also to be considered to include the end points of the range, unless expressly stated otherwise. For example, a range of “between 5 and 10,” “from 5 to 10,” or “5-10” should generally be considered to include the endpoints 5 and 10.

Further, when the phrase “up to” is used in connection with an amount or quantity, it is to be understood that the amount is at least a detectable amount or quantity. For example a material present in an amount “up to” a specified amount can be present from a detectable amount and up to and including the specified amount.

Silver Nanoparticles: 5, 25, 50, and 75 nm in diameter spherical silver nanoparticles (AgNPs) coated with polyvinylpyrrolidone (PVP; 89.3, 85, 76.5, 74% by mass respectively) and PVP-coated 15 nm gold nanoparticles (AuNPs; 91.6% PVP by mass) were purchased as dried nanopowders from nanoComposix, Inc (San Diego, Calif.). Nanoparticle dispersions were prepared by adding 1 ml of Phosphate Buffered Saline (PBS) (Invitrogen, Carlsbad, Calif.) to 20 mg of AgNPs or AuNPs in a 7 ml glass vial. The particles were dispersed by bath sonication. The resulting suspension was opaque and grey-brown in color. Silica-shelled AgNPs (Ag@SiNP), were purchased as an ethanol solution from nanoComposix, Inc. Prior to use, ethanol was exchanged with PBS using a size exclusion column. Citrate stabilized silver nanoplates were synthesized according to previously described methods. For cell culture experiments, the nanoparticles were diluted in cell culture media.

Cell Culture: MCF-7, MCF-10A, MDA-MB-231, BT-549, SUM-159, SK-LU-1, NCI-H358, OVCAR3, CAOV3, SKOV3 and A2780 cells were purchased from ATCC (Manassas, Va.) and expanded by the Comprehensive Cancer Center of Wake Forest University Cell Culture and Vector Core. LNCap and DU145 cells were a generous gift from Dr. Steven Kridel. RKO and H9-29 cells were a generous gift from Dr. Nicole Levi-Polyachenko. RKO and HT-26 cells were grown in McCoy's 5A supplemented with 1% penicillin and streptomycin and 10% FBS. MCF-7 cells were grown in DMEM/F12 supplemented with penicillin, streptomycin, L-glutamine, 10 μg/ml insulin, 10 ng/ml epidermal growth factor, 0.5 μg/ml hydrocortisone, and 10% fetal bovine serum. MCF-10A cells were grown in DMEM/F12 supplemented with penicillin, streptomycin, 2 mM L-glutamine, 5% HI-HS, 10 μg/ml insulin, 20 ng/ml epidermal growth factor, 0.5 μg/ml hydrocortisone, and 100 ng/ml Cholera toxin. MDA-MB-231 cells were grown in DMEM supplemented with 10% fetal bovine serum (vol:vol), 2 mM L-glutamine, penicillin (250 units/ml), and streptomycin (250 μg/ml) (all from Invitrogen). BT-549, NCI-H358, OVCAR3, CAOV3, SKOV3, A2780, LNCaP, and DU145 cells were grown in RPMI supplemented with 1% penicillin and streptomycin, and 10% FBS. SUM-159 cells were grown in HAM's F12 supplemented with 1% penicillin and streptomycin, 1% L-glutamine, 5% FBS, 5 μg/ml insulin, 1μg/ml hydrocortisone, and 10 μM HEPES. 184B5 cells were obtained and used with permission from Martha Stampfer (Lawrence Berkeley National Laboratory). 184B5 cells were cultured as previously described. hTERT iMEC cells were a generous gift from Dr. Elizabeth Alli, and were grown in DMEM/F12 supplemented with 10% FBS, 10 μg/ml insulin, 20 ng/ml hEGF, and 0.5 μg/ml hydrocortisone. Non-neoplastic HMT-3522 S1 (S1) mammary epithelial cells (or their neoplastic derivative HMT-3522 T4-2 (T4-2)) were cultured in H14 medium for 10 days, as described previously. Cells were cultured as monolayers in tissue culture treated plastics purchased from Corning Life Sciences (Lowell, Mass.) or on glass coverslips. Alternatively, S1, T4-2 and MDA-MB-231 cells were cultured in 4-well chamber slides (Millipore) in the presence of reconstituted basement membrane (Matrigel , Corning) to recapitulate the formation of polarized glandular structures (acini) and tumor nodules, respectively, as described

Dynamic Light Scattering: All measurements were made using the Zetasizer Nano ZS90 (Malvern Instruments, UK). AgNPs were sonicated for 5 min and then diluted to 1 mg/ml for 5 nm AgNPs or 40 μg/ml for all other AgNPs and 1 ml was added to a disposable, clear plastic cuvette (Sarstedt, Newton NC). Size measurements were taken in water (pH 5.5) or PBS (pH 7.4) using automatic settings. Zeta potential was measured in water using disposable folded capillary cells (Malvern Instruments, UK). Each measurement was taken in triplicate.

Nanoparticle Tracking Analysis: Measurements were made using the Nanosight NS500 (Malvern Instruments) at 25° C. AgNP dispersions (20 mg/mL) were diluted 1:50,000 in degassed Milli-Q (type I) water. The following settings were used for five measurements of preparations: NTA (nanoparticles tracking analysis) software version 3.1; camera shutter: 32 milliseconds; duration: 90; threshold: 4.

MTT assay: Cells were grown to log phase in their respective media, washed in PBS, trypsinized, and plated on 96-well plates at a density of 3,000-6,000 cells per well in 200 μL of complete media. Cells were allowed to recover for 18 hours and were then exposed to AgNPs for 48 or 72 hours. For NAC studies, cells were exposed to 4 mM NAC (Sigma Aldrich) diluted in growth media for 6 h prior to AgNP treatment. Media containing NAC was removed and replaced with media containing AgNPs. For induction of EMT, cells were exposed to 10 ng/ml TGF-β1 (R&D Systems) for 6 days at 37° C., prior to AgNP treatment. Media containing AgNPs were replaced with 200 μL of media containing 0.5 mg/mL MTT and incubated for 1 hour at 37° C. Medium was removed, and cells were lysed in 200 μL of DMSO and read using a Molecular Devices Emax Precision Microplate Reader at 560 nm and corrected for background at 650 nm.

ZEB1 Knockdown: BT549 cells were plated in 60mm tissue culture plates and allowed to grow to 80-90% confluence. Each plate was then transfected with a non-coding control shRNA plasmid, or a plasmid containing shRNA targeted against ZEB1. Knockdown constructs used in this study were obtained from Sigma Aldrich (Mission shRNA TRCN0000369267 and TRCN0000369266). Each plate was transfected using 1 μg plasmid in conjunction with Lipofectamine 2000 (Invitrogen) according to the manufacturer's protocol. The following day cells were reseeded and were maintained in medium containing puromycin (2 μg/ml). ZEB1 expression levels were verified using immunoblot prior to experimentation.

ICP-MS: MDA-MB-231 and MCF-10A cells were cultured as above in 60 mm tissue culture plates. Cells were then treated with AgNPs or vehicle (PBS) for 6 or 24 h. Cells were then trypsinized, washed twice in PBS, pelleted and stored at −20° C. Tumors and organas were minced and 200 mg of tissue was used for analysis. Samples were then digested with 10% HNO₃ using a microwave-assisted digestion system (Ethos UP, Milestone, Sorisole, Italy). The digested samples were then diluted to a final acid concentration of 2% v/v for tumors and 1% v/v for organs before Ag determination by inductively coupled plasma mass spectrometry (ICP-MS). Trace metal grade HNO₃ (Fisher, Pittsburgh, Pa., USA), and distilled-deionized water (18 MSΩ cm, Milli-Q®, Millipore, Bedford, Mass., USA) were used to digest the samples and prepare all solutions. Standard reference solutions used for calibration were prepared in 2% acid (HNO₃) for tumors or 1% acid for organs from a 1000 mg/L Ag stock (SPEX CertPrep, Metuchen, N.J., USA). A tandem ICP-MS (8800 Triple Quadrupole, Agilent, Tokyo, Japan) equipped with a SPS 4 automatic sampler, a Scott-type double pass spray chamber operated at 2 0C, and a Micromist concentric nebulizer was used in all determinations. Helium gas (≥99.999% purity, Airgas, Colfax, N.C., USA) was used in the ICP-MS's collision/reaction cell to minimize potential spectral interferences while monitoring the Ag isotope. Other relevant instrument operating conditions such as radio frequency applied power, sample depth, carrier gas flow rate, reaction gas flow rate, and the number of sweeps per replicate were 1550 W, 10.0 mm, 1.05 L/min, 4.0 mL/min, and 100, respectively.

Transmission Electron Microscopy: MDA-MB-231 or MCF-10A cells were cultured as above in 6-well tissue culture dishes. Cells were treated with AgNPs (150 μg/ml) for 1 h. All cells were washed thoroughly in PBS to remove AgNPs not bound or internalized by cells. Half of the wells were fixed in 2.5% glutaraldehyde at 4° C. overnight. Fresh cell culture media was added to the remaining wells which were incubated for 6 h more before fixation. Next, cells were scraped from the wells, pelleted, embedded in resin, cut into ultrathin sections (80 nm) and placed on copper coated formvar grids then imaged using a Tecnai Spirit transmission electron microscope (FEI). Samples were imaged without additional staining to facilitate the detection of AgNPs.

Western Blots: Cells were grown to log phase in their respective media, washed in PBS, trypsinized, and plated on 10 cm dishes at a density of 2×10⁶ cells in 10 mL of complete media. Cells were allowed to recover for 18 h and were then exposed to AgNPs for 6 or 24 h at 37 ° C. Medium was removed, and lysates were collected using M-PER Mammalian Protein Extraction Regent (78501, Thermo Scientific) supplemented with 1% Halt Protease & Phosphatase Inhibitor Cocktail (78440, Thermo Scientific). Protein concentration was determined for each sample using a bicinchoninic acid (BCA) protein assay kit (Thermo-Fisher/Pierce) according to the manufacturer's instructions. Proteins were size fractionated by gel electrophoresis and then transferred to a PVDF membrane. Nonspecific binding was blocked by incubation for 30 min at room temperature with Tris-buffered saline containing 5% powdered milk and 1% Triton X-100. Membranes were incubated overnight at 4 ° C. with primary antibodies (GRP78, PERK, phosphor-eIF2α, eIF2α, CHOP, ZEB1, Catalase, E-Cadherin, N-Cadherin, Vimentin, Slug or β-actin purchased from Cell Signaling Technologies, ESRP1 purchased from Sigma Prestige Antigens, or SOD2 purchased from Santa Cruz followed by incubation with polyclonal HRP-conjugated secondary antibodies (1:1000) for 1 hour at room temperature. Immunoreactive products were visualized by chemiluminescence (SuperSignal Femto West, Pierce Biotechnology) and quantified by densitometry using the Bio-Rad digital densitometry software.

Redox assays: Cells were grown and plated as described for MTT assay. Reduced glutathione (GSH) and oxidized glutathione were quantified using the Promega GSH-Glo Glutathione Assay according to the manufacturer's instructions. Reduced nicotinamide adenine dinucleotide phosphate (NADPH) and its oxidized form (NADP+) were quantified using the Promega NADP/NADPH-Glo Assay according to the manufacturer's instructions. Luminescence was read using a Tecan GENios microplate reader.

ROS Detection: Cells (0.5-1.0×10⁵ cells) were seeded in 24-well tissue culture plates and allowed to attach overnight. The following day cells were treated as indicated for 24 hours at 37° C. Medium was removed, cells were washed with PBS (with magnesium and calcium), and incubated with 10 μM 2′,7′-dichlorodihydrofluorescein diacetate (H₂DCF-DA) (Invitrogen) diluted in PBS (with calcium and magnesium) for 5 min at 37° C. Cells were imaged using the EVOS FL Auto (Thermo Scientific).

Flow cytometry: Cells were grown to log phase in their respective media, washed in PBS, trypsinized, and plated on 10 cm dishes at a density of 1.25×10 cells for control plates or 2×10 cells for treatment plates and were allowed to adhere for 18 h. Cells were treated with 25 nm 0-1,000 μg/m1 AgNPs (nanoComposix, San Diego, Calif.) for 24 h. Cells were harvested as described and APC Annexin V and propidium iodide staining was performed as per the manufacturer's instructions (BD Pharmingen; San Diego, Calif.). Briefly, cells were trypsinized, pelleted, washed twice with cold PBS, and then suspended in 1× Annexin V binding buffer at a concentration of 1×10 cells/ml. 1×10 cells were then mixed with Annexin V and incubated for 15 minutes at room temperature in the dark. Four hundred microliters of 1× Binding Buffer (BD Biosciences, East Rutherford, N.J.) was added with or without propidium iodide (2.0 ug/ml; final concentration). Labeled cells were analyzed on the Accuri6 Flow Cytometer (BD Biosciences, East Rutherford, N.J.). Analysis of data was performed using FCS Express version 3 (De Novo Softwar, Los Angeles, Calif.). For cell cycle analysis, cells were treated as indicated, fixed in 50% ice-cold ethanol, washed once in PBS, and then were treated with FxCycle PI/RNase staining solution (Life Technologies) per the manufacturer's protocol. Analysis was performed using ModFit software.

Immunofluorescence: Cells were permeabilized for 20 minutes with 0.5% triton X-100 in cytoskeleton buffer (100 mM NaCl, 300 mM sucrose, 10 mM PIPES pH 6.8, 5 mM MgCl2, 10 μg/m1 aprotinin [Sigma-Aldrich], 1 mM 4-(2-aminoethyl)-benzenesulfonyl fluoride, hydrochloride [Roche Diagnostics], and 250 μM NaF) and fixed with 4% paraformaldehyde. After blocking with 10% goat serum in immunofluorescence buffer (130 mM NaCl, 13 mM Na₂HPO₄, 3.5 mM NaH₂PO₄, 7.7 mM NaN₃, 0.1% BSA, 0.2% triton X-100, 0.05% tween 20), cells were incubated with the following antibodies (overnight, 4° C.): 53BP1 (AbCam, Ab36823), γH2AX (clone JBW301, Millipore), Ki67 (Thermo Scientific, PAS-19462), β4-integrin (Millipore, MAB1964), or ZO-1 (clone 1Al2, Invitrogen). Primary antibodies were detected with secondary antibodies coupled to Alexa Fluor® 488 or Alexa Fluor® 568 or (Life Technologies) incubated in blocking buffer 40 minutes at ambient temperature. DNA was counterstained with 4′,6-diamidino-2-phenylindole (DAPI). Fluorescent signals were imaged with a Zeiss CLSM710 confocal microscope using a 63× oil (NA=1.4) objective. 53BP1 and γH2AX repair foci were quantified by visual scoring on confocal images (3D cultures). The percentage of S1 acini with apical polarity was determined by visual scoring of ZO-1 signals using an Olympus IX83 microscope equipped with a 60× oil (1.35 NA) lens. A Cs-137 irradiator (IL Sherpherd; Mark I-68A) was used for cell irradiation (3 Gy).

In vivo studies: All animal experiments were performed with prior approval by Wake Forest University Institutional Animal Care and Use Committee. Female, 8-10 week old nu/nu mice were purchased from the Charles River Labs. Mice were housed in groups of five in individually ventilated cages with a 12 h light/dark cycle and were allowed access to food and water ad libitum. Mice were allowed to acclimatize for 2 weeks prior to beginning experiments. For tumor inoculation, MDA-MB-231 cells in Matrigel (BD Biosystems) (50 μl containing 2>10 cells) were injected into the fourth inguinal mammary fat pad of mice. Tumor growth was monitored by calipers and volume was determined using the formula: volume=0.52×(width)×(length)×(width+length)/2 where length and width are the two largest perpendicular diameters. When the tumors reached an average volume of 100 mm (approximately 3 wk post-implantation), mice were weighed and randomized into two groups of 8. Mice were injected intravenously in the lateral tail vein, 3 times/wk for 10 wk, with AgNPs in PBS (6 mg/kg) or PBS alone. Tumor growth, body weight and general health were monitored over time. For biodistribution studies, mice were injected intravenously in the lateral tail vein with a single dose of AgNPs in PBS (6 mg/kg) or PBS alone. Organs were collected after 24 h.

### The Cytotoxicity Profile of AgNPs is Selective for CLBC and Independent of Particle Size, Shape or Capping Agent

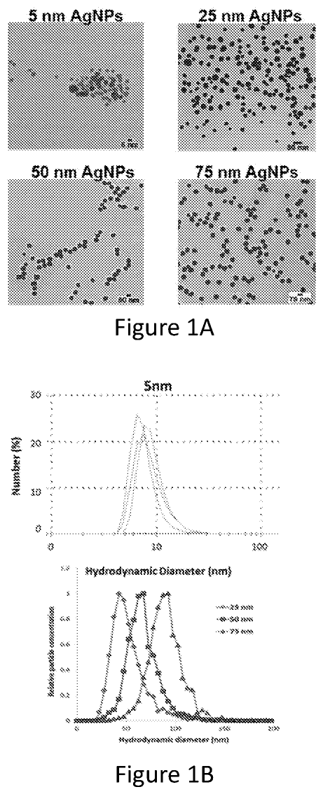

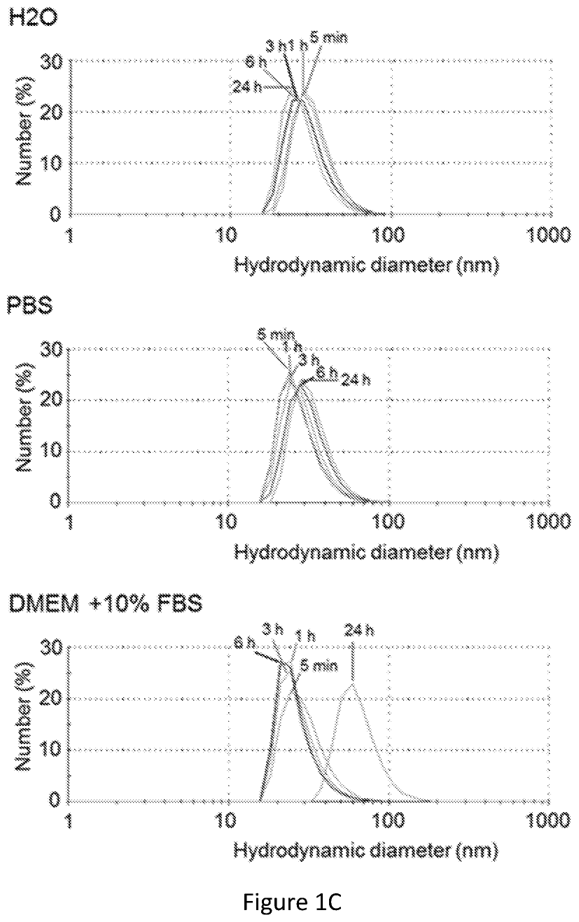

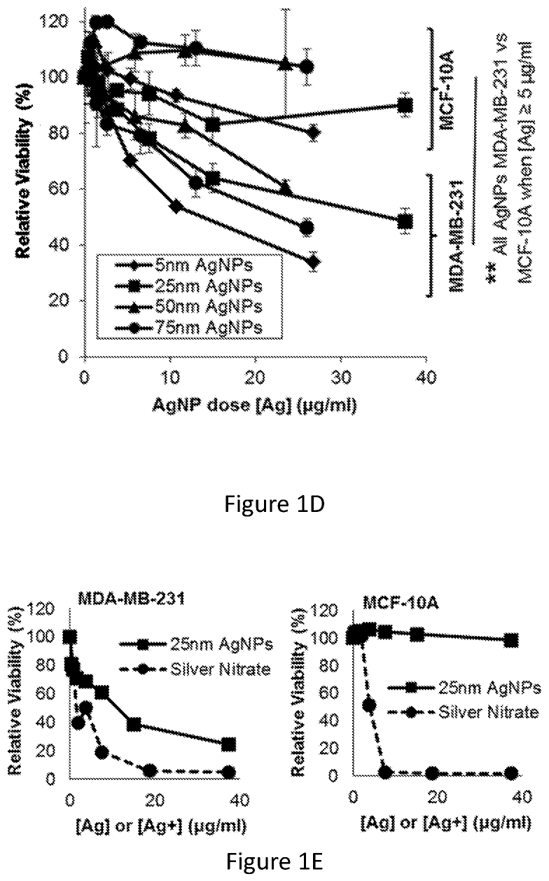

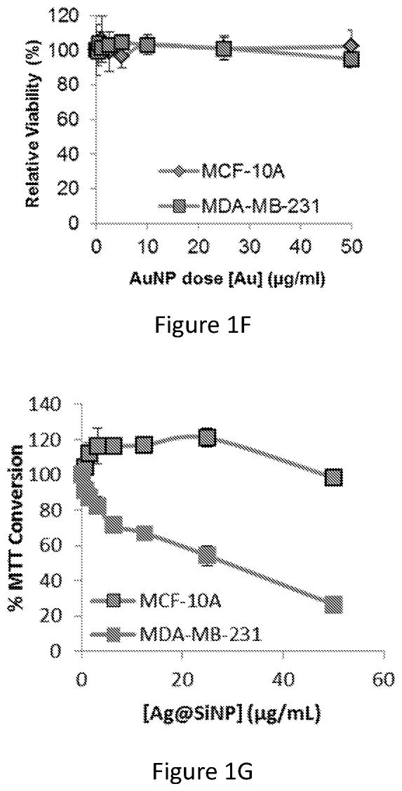

Monodisperse AgNPs of increasing diameter (5 nm, 25 nm, 50 nm, 75 nm) stabilized with a high (>74% by mass) percentage of polyvinylpyrrolidone (PVP) as a capping agent were used to determine if AgNP size influences the TNBC-selective cytotoxicity previously observed using a heterogeneous dispersion of AgNPs. The manufacturer provided data regarding particle size by TEM (FIG. 1A), which was verified using both Nanoparticle Tracking Analysis (NTA) and Dynamic Light Scattering (DLS) as shown in FIG. 1B.

All AgNPs were monodispersed and possessed negative ζ-potentials in water at pH 6.5 (Table I). The colloidal stability over time of 25 nm AgNPs was further evaluated via DLS (FIG. 1C). No change in hydrodynamic diameter was observed in water or PBS (pH 7.4) over 24 h, but an increase in hydrodynamic diameter was observed after storage of AgNPs in cell culture media (DMEM) containing 10% serum. The particles remained well suspended without evidence of precipitation, and it is likely that this increase was due to the formation of a protein corona.

TNBC cells (MDA-MB-231) and non-cancerous mammary epithelial cells (MCF-10A) were exposed to increasing concentrations of various sizes of AgNPs and evaluated for cytotoxicity 48 h later. AgNPs were significantly (p<0.01) more cytotoxic toward MDA-MB-231 cells compared to non-cancerous MCF-10A cells for all particle sizes at silver concentrations of 5 μg/ml or greater. All sizes of AgNPs exhibited similar cytotoxicity (no significant differences; (FIG. 1D) toward MDA-MB-231 cells. Release of silver ions (AO from AgNPs has been reported to contribute to their cytotoxicity. However, the TNBC selective cytotoxic effects that were observed were mediated by intact AgNPs. As shown in FIG. 1E, Ag⁺ (AgNO₃) was highly cytotoxic to both MDA-MB-231 and MCF-10A. In contrast, MCF-10A cells were resistant to AgNPs while MDA-MB-231 cells were sensitive to AgNPs. In addition, PVP-coated, 15 nm gold nanoparticles (AuNPs) were not cytotoxic toward either MDA-MB-231 or MCF-10A cells (FIG. 1F), further confirming that the TNBC selective cytotoxicity profile was specific to AgNPs and is a property that is unshared among all NPs composed of heavy metals.

To determine if AgNP shape or coating affects the TNBC-selective cytotoxicity observed for spherical PVP-coated AgNPs, the cytotoxicity of silica-shelled AgNPs was assessed (FIG. 1G). These particles also were highly cytotoxic to MDA-MB-231 cells but not to MCF-10A cells. The cytotoxicity of methoxypolyethyleneglycol-coated spherical AgNPs (mPEG-AgNPs) and citrate-stabilized, triangular silver nanoplates (AgNPPs) were also prepared, characterized, and assessed. AgNPs possess a strong affinity for sulfhydryl (thiol) groups, which can be exploited to functionalize AgNPs with different capping agents or targeting moieties. 5 nm PVP-coated AgNPs obtained from nanoComposix Inc. were functionalized with methoxypolyethyleneglycol_{5,000}-thiol (mPEG-SH). The PVP-thiol exchange was monitored via ultraviolet/visible (UV/Vis) spectroscopy, and a dampening of the absorbance peak at ˜405 nm and a red shift was observed for the mPEG-AgNPs compared to the PVP-AgNPs. Additionally, there was an increase in hydrodynamic diameter and a more positive -potential for the mPEG-AgNPs. MDA-MB-231 cells were significantly more sensitive than MCF-10A cells to these NPs, indicating that the TNBC-selective cytotoxicity is independent of capping agent. Citrate-stabilized AgNPPs were generally uniform in size and shape and possessed a mean hydrodynamic diameter of approximately 133 nm. In contrast to the negatively charged PVP-coated particles, they possessed a cationic (+27 mV) -potential. Notably, MDA-MB-231 cells were significantly more sensitive than MCF-10A cells to these particles, indicating that the TNBC-selective cytotoxicity of AgNPs was independent of coating, capping agent, shape or size.

TABLE I
Physicochemical characterization of AgNPs

| | | | DLS hydrodynamic | ζ-potential ± |
|---|---|---|---|---|
| Nanoparticle | % mass | % mass | diameter ± standard | standard deviation |
| core diameter | Ag | PVP | deviation (nm) | (mV) |
| 5 nm | 10.7% | 89.3% | 5.5 ± 1.6 | −12.7 ± 0.86 |
| 25 nm | 15-25.6% | 74.4-85% | 44.4 ± 12.8 | −14.24 ± 0.80 |
| 50 nm | 23.5% | 76.5% | 66.3 ± 8.8 | −46.4 ± 0.44 |
| 75 nm | 26% | 74% | 91.2 ± 9.1 | −38.4 ± 0.80 |

Core particle size and Ag and PVP content are reported by the manufacturer. Particle size and potential were determined after dispersing the AgNPs in water at pH 6.5 and determined using the Malvern Zetasizer Nano ZS. Triplicate measurements were taken for each sample.

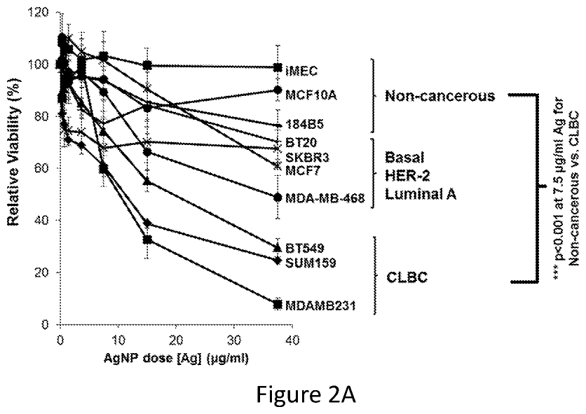

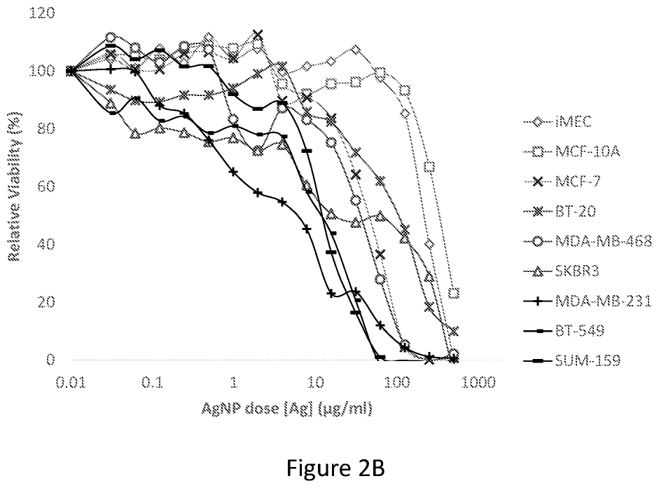

Next, the cytotoxicity of 25 nm AgNPs in a panel of breast cell lines was determined. TNBC cell lines which can be further classified as the CLBC (MDA-MB-231, BT-549, SUM-159) or basal (BT-20, MDA-MB-468) molecular subtypes were tested, as were luminal A (MCF-7), HER-2+ (SKBR3), and non-cancerous breast (MCF-10A, 184B5, iMEC) cell lines (FIG. 2A). The AgNPs showed the greatest cytotoxicity toward a subset of TNBC cell lines, the CLBC cell lines. AgNPs were less cytotoxic in other breast cancer subtypes (basal, luminal A, HER-2) and significantly less cytotoxic at 7.5 μg/ml Ag in non-cancerous breast cell lines after a 48 h treatment (p<0.001 for all comparisons at a silver metal concentration of 7.5 μg/ml or greater). A panel of cancerous and non-cancerous breast cell lines was exposed to 25 nm AgNPs for 72 h and the IC50 was calculated for each cell line (FIG. 2B and Table II). As with the 48 h treatment, the CLBC cell lines were the most sensitive to AgNPs following a 72 h treatment.

TABLE II

| | | IC₅₀ [AgNP] | IC₈₀ [Ag] |
|---|---|---|---|
| Cell Line | Molecular Subtype | (μg/ml) | (μg/ml) |
| MCF-10A | Normal | 1006.2 | 251.55 |
| iMEC | Normal | 555.17 | 138.79 |
| MCF-7 | Luminal A | 110.29 | 27.57 |
| BT-20 | Basal | 376.7 | 188.35 |
| MDA-MB-468 | Basal | 113.97 | 28.49 |
| SKBR3 | HER-2+ | 162.02 | 40.50 |
| SUM-159 | CLBC | 62.95 | 16.95 |
| BT-549 | CLBC | 24.54 | 6.14 |
| MDA-MB-231 | CLBC | 17.92 | 4.48 |

Collectively, these results establish that the CLBC-selective cytotoxic properties of AgNPs are independent of particle size, shape or capping agent. The CLBC-specific cytotoxicity of AgNPs is not shared by ionic silver, demonstrating one of the first examples of a “new to nano” cytotoxic property. Because all types of AgNPs exhibited similar cytotoxicity profiles, in subsequent studies the inventors focused on the 25 nm PVP-coated AgNPs, as they are already reliably mass produced and are large enough to avoid rapid urinary clearance, yet small enough to evade clearance by the mononuclear phagocyte system and accumulate in tumors due to the EPR effect.

It should be understood that any silver nanoparticle not inconsistent with the goal of the disclosure is contemplated. In some embodiments, silver nanoparticles, as described herein, are monodispersed and free of aggregation. For example, in some cases, silver nanoparticles can have one or more coatings, shells, functional side groups, shapes, sizes, and/or capping agents and are monodispersed. In some instances, the one or more coatings, shells, functional side groups, shapes, sizes, and/or capping agents can prevent aggregation of the silver nanoparticles.

Furthermore, it should be understood that the silver concentration of silver nanoparticles described herein, is determined independently of wherein the nanoparticle the silver resides. For example, the silver can reside in a core or a shell of the nanoparticle, or both. Wherein the silver resides in a shell, the nanoparticles can have more than one shell. For example, a silver nanoparticle having one or more shells can have one or more shells comprising silver.

Furthermore, it should be understood that the diameter of a nanoparticle, in some embodiments, includes only the core of the nanoparticle. In other embodiments, a diameter includes both a core and a shell of the nanoparticle, wherein the shell represents a coating of the nanoparticle. In some cases, the diameter is an average diameter of nanoparticles disposed in a suspension. The average diameter can be determined by NTA and/or DLS analysis. In some cases, the diameter of nanoparticles is dynamic and can fluctuate while in suspension.

### Intravenous Delivery of AgNPs is Safe and Effective for Treatment of CLBC Xenografts In Vivo

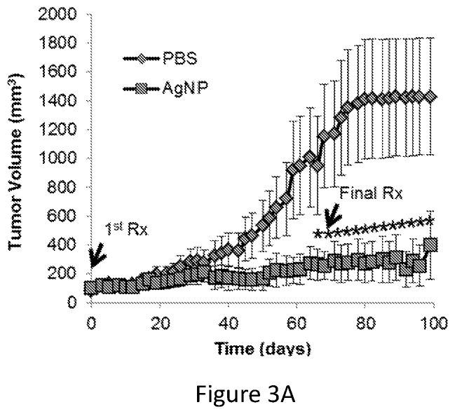

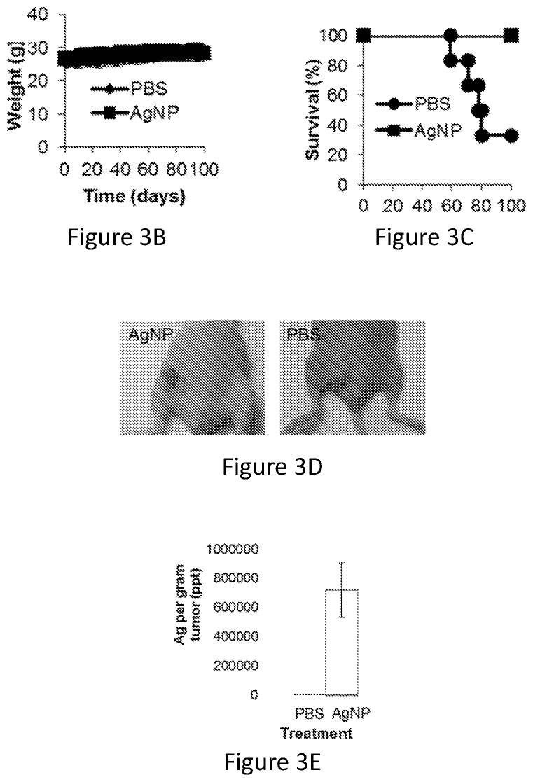

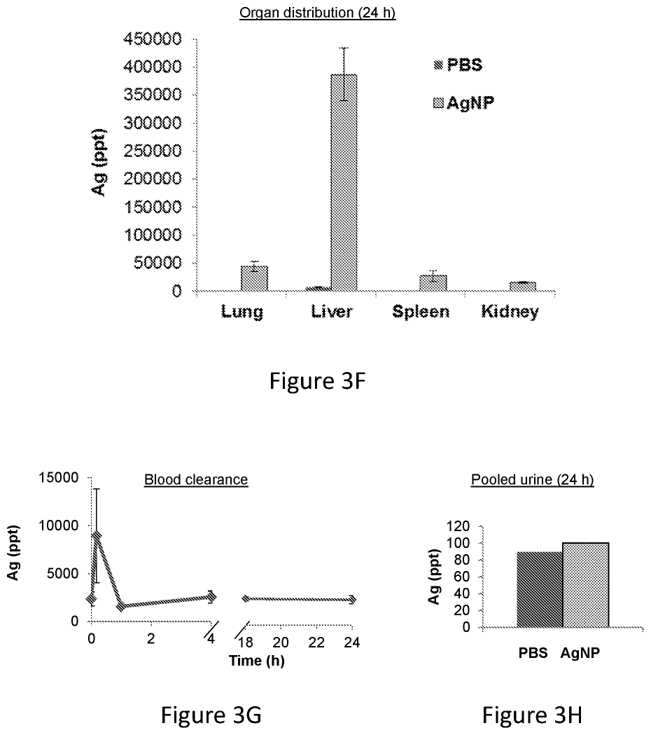

An in vivo dose escalation study was performed in nude mice. The maximum tolerated dose (MTD) for the 25 nm diameter, PVP-capped AgNP formulation was 9-12 mg/kg following a single, bolus intravenous injection (not shown). Subsequently, nude mice bearing orthotopic MDA-MB-231 tumors implanted in the mammary fat pad were injected intravenously with the lead 25 nm, PVP-stabilized AgNPs at ⅔ MTD (6 mg/kg) or PBS three times per week for 10 weeks. Intravenous injection of AgNPs significantly reduced CLBC tumor growth in mice (FIG. 3A). Throughout the study, mice were monitored for signs of toxicity including weight loss, hunched posture/immobility, and rapid or shallow breathing. No difference in weight between PBS and AgNP treatment mice was observed (FIG. 3B), nor were overt signs of toxicity noticeable, indicating that these NPs were potentially both effective and safe. All mice treated with AgNPs survived for the duration of the study (100 days) while only ⅓ of PBS group survived (FIG. 3C). Treatment caused tumor necrosis as evidenced by scabbing of the tumor as the treatment progressed (FIG. 3D). After 100 days, all AgNP treated mice and surviving control animals were euthanized and the amount of silver remaining in the tumors was quantified by ICP-MS. As shown in FIG. 3E, silver remained detectable within the tumor/surrounding necrotic tissue of mice treated with AgNPs.

An in vivo biodistribution study was performed in nude mice following a 24 h single bolus dose intravenous injection of 6 mg/kg AgNPs. Mice were euthanized and organs were collected after 24 h and analyzed by ICP-MS for silver content. After 24 h, the largest amount of Ag found was in the liver, with lower detectable levels found in the lungs, spleen, and kidneys (FIG. 3F). Minimal Ag was found in the urine (FIG. 3G), which corresponds with the low levels of Ag detected in the kidneys.

### AgNPs Traffic to Different Intracellular Compartments After Uptake by TNBC Compared to Non-Cancerous Breast Cells

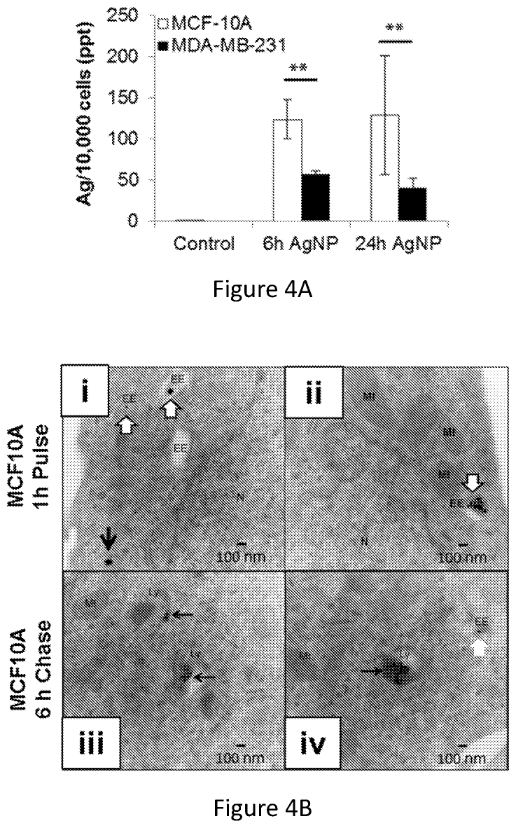

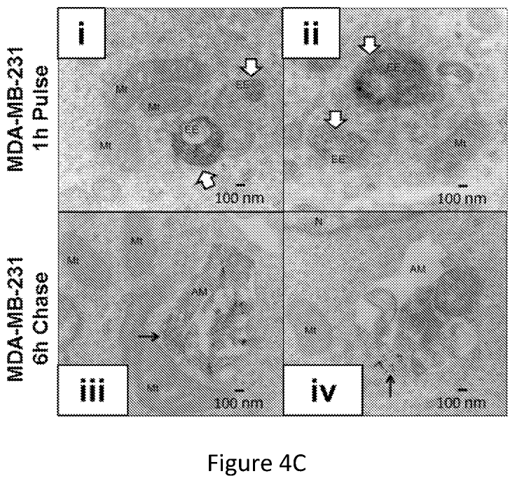

NP formulations of silver may function as a “Trojan Horse”, carrying silver metal (Ag) across cell membranes and influencing the rate, extent, location and/or timing of Ag+ release. The cell uptake and intracellular trafficking of AgNPs was examined. MDA-MB-231 and MCF-10A cells were exposed to equal amounts of AgNPs for 6 or 24 h, washed extensively, counted and Ag content was quantified by ICP-MS. Notably, MCF-10A cells bound or took up more than twice as much Ag as MDA-MB-231 cells, and most of the Ag became cell associated during the first 6 h of exposure (FIG. 4A). Thus, the greater sensitivity of MDA-MB-231 cells to AgNPs was not due to these cells taking up more NPs than the relatively insensitive MCF-10A cells.

To study intracellular trafficking of AgNPs, cells were pulsed with AgNPs for 1 h to allow for AgNP uptake then washed and fixed or chased for 6 h before fixing to allow for AgNPs to traffic within cells. Imaging by transmission electron microscopy (TEM) confirmed that MCF-10A cells took up more AgNPs than MDA-MB-231 cells. Intact AgNPs could clearly be seen on the surface and within endosomes of MCF-10A cells after 1 h exposure to AgNPs (FIG. 4B i and ii). In contrast, fewer AgNPs were found within endosomes in MDA-MB-231 cells (FIG. 4C i and ii) and only rarely were AgNPs found on the surface of these cells. Higher power images of AgNPs in MCF-10A cells confirmed that intact nanoparticles are in endosomes after 1 h, which appear to fuse with electron dense lysosomes within 6 h (FIG. 4B). After 1 h, AgNPs were also found in endosomes of MDA-MB-231 cells, but the particles are notably degraded in comparison to those found in MCF-10A cells (FIG. 4C i and ii). Importantly, after 6 h, degraded AgNPs were found in autophagic vesicles consistent with amphisomes (FIG. 4B iii and iv), which are formed by fusion of late endosomes and autophagosomes.

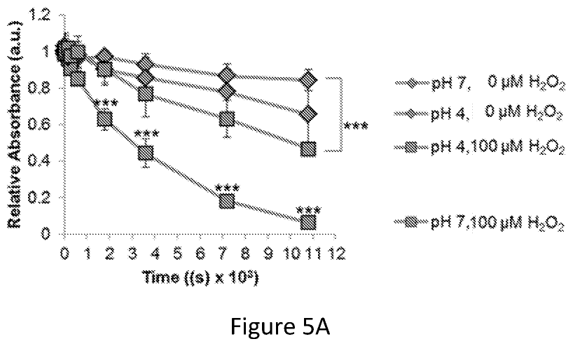

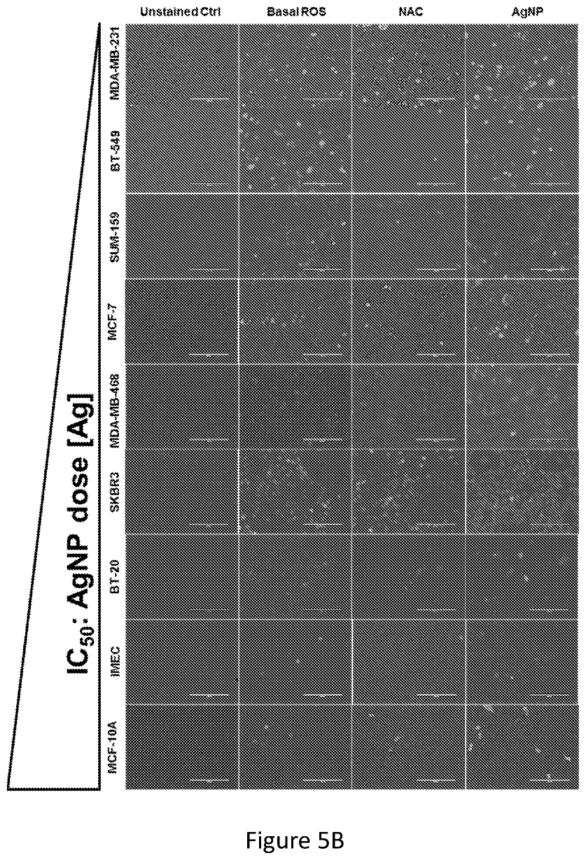

It is possible that intracellular reactive oxygen species (ROS) liberate Ag⁺ ions from AgNPs. The released Ag⁺ would then induce damage and eventually cause cell death. Notably, the dissolution of AgNPs by hydrogen peroxide (H₂O₂), a major component of cellular ROS, is reported to exhibit pH dependence. At neutral or basic pH, the degradation of AgNPs is expected to be rapid while at acidic pH, H₂O₂ mediated degradation is slow. As shown in FIG. 5A, AgNPs are stable at pH 7, but rapidly degrade when exposed to H₂O₂. In contrast, the degradation of AgNPs by H₂O₂ at pH 4 is considerably slower. From TEM images (FIG. 4),

AgNPs appear to degrade within endosomes at pH 7 immediately upon internalization by MDA-MB-231 cells. In contrast, AgNPs remained intact within endosomes of MCF-10A cells, and eventually translocated into acidic lysosomes. It is possible that the high basal levels of ROS observed in the MDA-MB-231 cell line led to the increased dissolution of AgNPs observed by TEM. This may contribute to the CLBC-selective cytotoxicity of AgNPs by causing rapid ionization of the AgNPs. In contrast, the low ROS environment of MCF-10A and other AgNP-insensitive cells would not trigger the dissolution of AgNPs.

Basal levels of ROS in a panel of breast cell lines and the effect of AgNP exposure on ROS were examined using the ROS sensitive dye, H₂DCF-DA (FIG. 5B). MDA-MB-231 cells exhibited high basal ROS activity in comparison to MCF-10A cells. There was a correlation between increasing ROS levels and increasing sensitivity to AgNPs. Cell lines that were the most sensitive to AgNP treatment, predominately CLBC cell lines, exhibited high basal levels of ROS, whereas AgNP insensitive cell lines demonstrated low basal levels of ROS. 24 h 10 mM N-acetylcysteine (NAC) treatment was sufficient to partially mitigate some of the basal ROS in the AgNP sensitive cell lines. In response to AgNP exposure, there was a slight increase in ROS detected in the sensitive cell lines; however, high basal levels in these cells prevented detection of significant ROS increases. In the AgNP insensitive cell lines, AgNP exposure increased ROS in the luminal A (MCF-7) and basal-like (MDA-MB-468, BT-20) cell lines, whereas there was no detectable increase in ROS in the non-cancerous breast cell lines (MCF-10A, iMEC). It is likely that elevated levels of ROS in CLBC cell lines cause rapid intracellular dissolution of AgNPs, and the subsequent release of Ag⁺ induces cell death. In contrast, the lower levels of ROS present in non-cancerous breast cells are insufficient to trigger the rapid dissolution of AgNPs.

In some embodiments, a method of treating cancer further comprises dissolution and/or degradation of the silver nanoparticles. For example, in some cases, the silver nanoparticles are degraded and/or dissolved in or among the cancerous cell population. In some instances, degradation and/or dissolution of the silver nanoparticles is performed by reactive oxygen species, such as peroxides, superoxide, hydroxyl radical, or singlet oxygen, present in or among the cancerous cell population.

In other embodiments, a method of determining response of a cancerous cell population comprises characterizing a redox state of the cancerous cell population. Characterizing the redox state, in some embodiments, includes quantifying a level of one or more reactive oxygen species in or among the cancerous cell population. For example, reactive oxygen species, such as peroxides, superoxide, hydroxyl radical, or singlet oxygen, can be quantified. In other embodiments, characterizing the redox state can include determining the pH and/or oxygen concentration.

AgNPs Induce Redox Imbalance and Cytotoxicity in CLBC, Which Can be Mitigated Via NAC Pre-Treatment, but is Independent of SOD2 or Catalase Expression

AgNPs induced oxidative damage to protein thiols in TNBC cells without causing comparable damage to luminal breast cancer or non-cancerous breast cells. The tripeptide non-protein thiol, glutathione (GSH), plays a key role in mitigating oxidative damage, and can modulate protein synthesis and folding, signal transduction, and cell proliferation. In the presence of ROS, GSH is oxidized to form a homodimer disulfide (GSSG). Nicotinamide adenine dinucleotide phosphate (NADPH) also protects against oxidative stress and provides reducing equivalents allowing the regeneration of reduced GSH from its oxidized disulfide form (GSSH). Additionally, NADPH is necessary for lipid and nucleic acid synthesis. Therefore, substances causing imbalances in the redox balance of GSH/GSSG and NADPH/NADP⁺ may impact normal cell function, even at non-lethal doses.

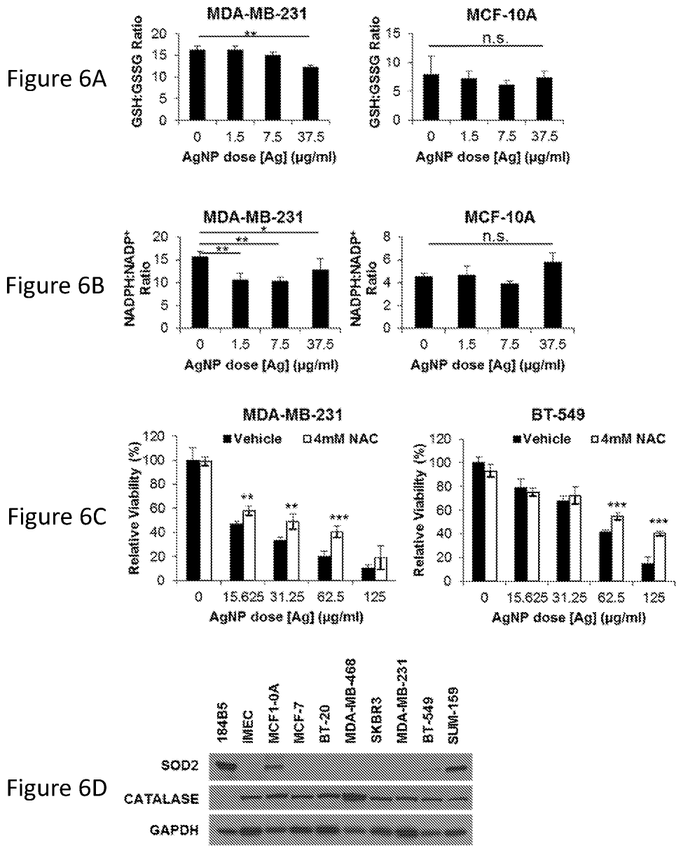

To determine the effect of AgNPs on the redox state of CLBC and non-CLBC cells, the ratio of the oxidized and reduced forms of these antioxidants were quantified in MDA-MB-231 and MCF-10A exposed to AgNPs. As shown in FIG. 6A and FIG. 6B, AgNPs decreased both GSH/GSSG and NADPH/NADP⁺ ratios in MDA-MB-231 cells, but not in MCF-10A cells. This indicated that AgNPs perturbed the redox balance in the CLBC cells at doses that had no effect on non-cancerous breast epithelial cells. As shown in FIG. 5, MDA-MB-231 and BT-549 CLBC cells possess high basal levels of ROS and are the most sensitive to AgNP treatment (FIG. 2B and Table II). Both cell lines were pre-treated with 4 mM NAC for 6 h to decrease basal levels of ROS, prior to a 48 h AgNP treatment. NAC pre-treatment significantly decreased cytotoxicity of AgNP treatment at Ag concentrations of 15.625-62.5 μg/ml in MDA-MB-231 cells and 62.5-125μg/ml in BT-549 cells. This further suggests that high basal levels of ROS in CLBC cells contribute to AgNP-mediated cytotoxicity. Manganese superoxide dismutase (SOD2) and catalase are antioxidant enzymes that play roles in mitigating oxidative stress by converting free radicals to H₂O₂ and H₂O₂ to water and oxygen respectively. SOD2 and catalase have been implicated in tumor progression and metastasis in several cancers due to their ability to allow cancers to survive in pro-oxidative environments. SOD2 and catalase expression was assessed via western blot in a panel of breast cancer and non-cancerous breast cell lines, which demonstrated that AgNP-induced cytotoxicity was independent of SOD2 or catalase expression (FIG. 6D).

### AgNPs Induce ER Stress, Apoptosis, and Slowing Through S-Phase in CLBC Cells Without Affecting Non-Cancerous Breast Epithelial Cells

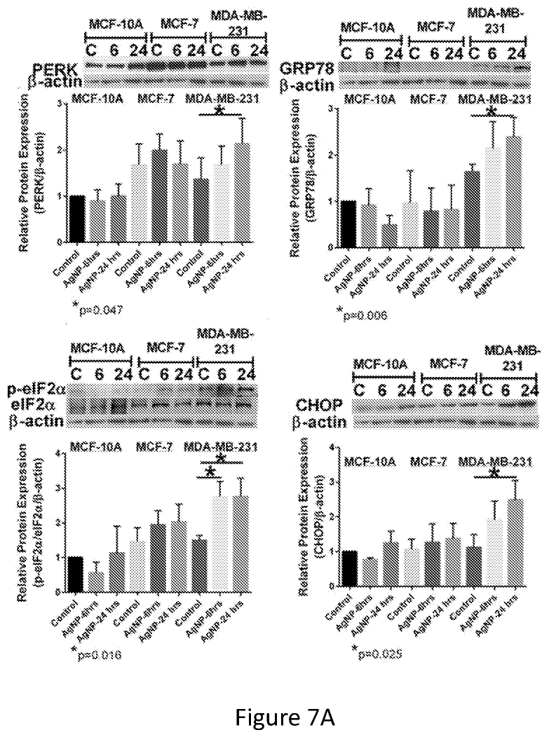

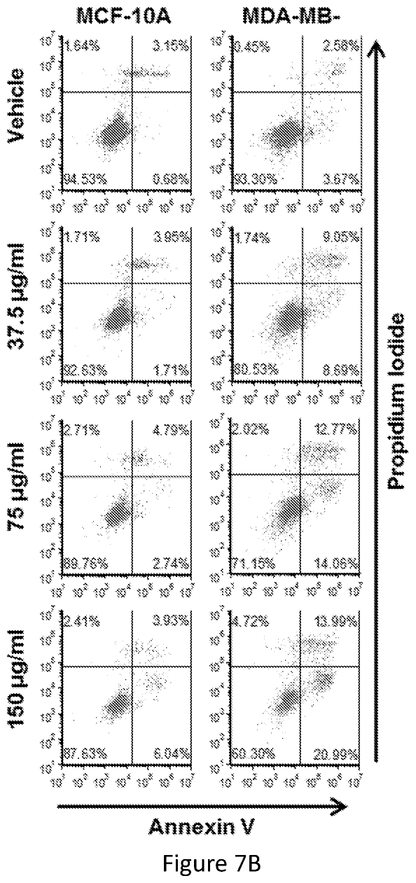

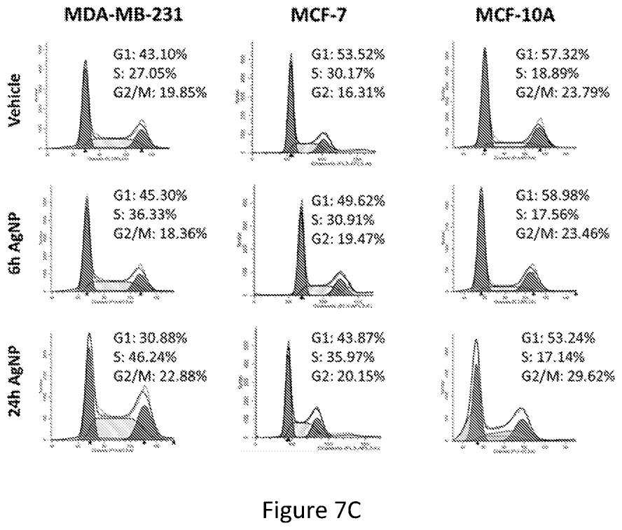

AgNPs can induce ER stress in vitro and in vivo, but whether CLBC or other cancer cells are more sensitive the AgNP-induced ER stress than equivalent non-cancerous cells has not been reported. The effect of AgNPs on ER stress in MCF-10A and MDA-MB-231 cells was then determined. In addition, the effect of AgNPs on ER stress in luminal A MCF-7 breast cancer cells was examined. Cells under ER stress activate the PERK (protein kinase R-like ER kinase) signaling pathway. PERK activation leads to phosphorylation of eukaryotic translation initiation factor 2α (eIF2α or p-eIF2α when phosphorylated) and increased synthesis of the ER-chaperone protein GRP78 (78 kDa glucose-regulated protein). Failure to mitigate ER stress leads to synthesis of the pro-apoptotic protein CHOP (C/EBP homologous protein). Therefore, PERK, total eIF2α and p-eIF2α, GRP78, and CHOP expression was quantified by western blot after 6 or 24 h treatment of cells with AgNPs (FIG. 7A). After exposure to AgNPs, no significant change in PERK, p-eIF2α/total eIF2α ratio, GRP78, or CHOP expression was found for MCF-10A or MCF-7 cells at either treatment time, indicating that ER stress was not induced. In contrast, AgNPs induced ER stress in MDA-MB-231 as indicated by an increase in PERK, p-eIF2α/eIF2α ratio, GRP78, and CHOP.

The increased CHOP expression observed in CLBC cells treated with AgNPs is expected to induce apoptosis. To determine the mechanism of cell death caused by AgNPs, annexin V (AnnV) and propidium iodide (PI) co-staining was performed on the adherent population of non-cancerous MCF-10A breast cells and MDA-MB-231 CLBC cells following a 48 h treatment with increasing doses of 25 nm AgNPs. This method helps distinguish between necrosis/complete loss of membrane integrity (PI+, AnnV-), early apoptosis (PI-, AnnV+), and late apoptosis (PI+, AnnV+). AgNPs induced a dose-dependent increase in early-stage apoptosis, late-stage apoptosis and necrosis in MDA-MB-231 compared to vehicle control which correlated with 24 h viability assay data (FIG. 7B). Conversely, AgNPs had a minimal effect on early-stage, late-stage apoptosis and necrosis in MCF-10A. These results indicate that in CLBC cells, but not in non-CLBC cells, AgNPs cause activation of the UPR, synthesis of the pro-apoptotic protein, CHOP, and induction of apoptosis.

Thapsigargin is a chemical inducer of ER stress which has been shown to activate the PERK arm of the UPR in MDA-MB-231 cells. GSK2606414 is the first-described small molecule selective inhibitor of PERK. MDA-MB-231 cells were treated with 1 μM of the ER stress inducer, thapsigargin, 25 nM of the PERK inhibitor (PERKi), GSK2602414, or the combination of thapsigargin and GSK2606414 for 4 h to verify the efficacy of the PERKi, GSK2606414. Immunoblotting confirmed that thapsigargin was sufficient to induce activation of the PERK arm of the UPR as evident by an increase in phosphorylation of the downstream effector, eIF2α (p-eIF2α). Co-treatment with the PERKi, GSK2606414, and thapsigargin decreased p-eIF2α expression compared to thapsigargin alone. This indicates that the PERKi, GSK2606414, is sufficient to mitigate PERK arm activation as an indicator of ER stress. To further evaluate the role of ER stress in AgNP-induced cytotoxicity, MDA-MB-231 cells were treated with increasing doses of AgNPs alone or in combination with the PERKi, GSK2606414, for 48 h to determine if PERK inhibition provides protection against AgNP-induced cytotoxicity. There was no significant difference in viability observed via MTT assay between cells treated with AgNPs alone or in combination with the PERKi. This indicates that while AgNPs do induce ER stress via activation of the PERK arm of the UPR and likely plays a role in the apoptotic cell death observed in CLBC cells (FIG. 7A and B), ER stress mediated by the PERK arm of the UPR is not the only cause of AgNP-mediated cell death.

In parallel to the ER stress studies, the effect of AgNP treatment on the cell cycle was examined for MDA-MB-231, MCF-7, and MCF-10A cell lines. As shown in FIG. 7C, treatment of MDA-MB-231 cells with AgNPs induced a time dependent decrease in the number of cells in G1 and an increase in S-phase cells. A similar, though less dramatic effect on cell cycle was observed for MCF-7 cells. In contrast, there was little effect on the cell cycle distribution of MCF-10A cells treated with AgNPs. G1 and S-phase accumulation correlated with AgNP sensitivity in all cell lines tested.

### AgNPs Induce DNA Damage and Apoptosis in 3D Culture of CLBC Cells But Not in 3D Culture of Non-Neoplastic Mammary Epithelial Cells

Because an increase of cells in S-phase is indicative of DNA damage, evidence of DNA damage and apoptosis following AgNP treatment was sought in 3D models of CLBC and normal-like breast cells. The breast epithelium consists of glandular structures (acini) connected to a branched ductal system. The architecture of the acini and the ducts is characterized by a central lumen, apical and lateral cell-cell junctional complexes (including apical tight junctions (TJ)), and hemidesmosomes ligating the basement membrane at the basal side of the gland/duct. The establishment and maintenance of apical-basal polarity is essential for homeostasis and can be recapitulated by placing epithelial cells in well-defined 3D culture conditions. Notably, non-neoplastic mammary epithelial cells develop growth-arrested, polarized spherical structures similar to acini in vivo when cultured with reconstituted basement membrane (Matrigel®). Under similar culture conditions, cancer cells fail to growth-arrest and develop disorganized masses reminiscent of tumor nodules.

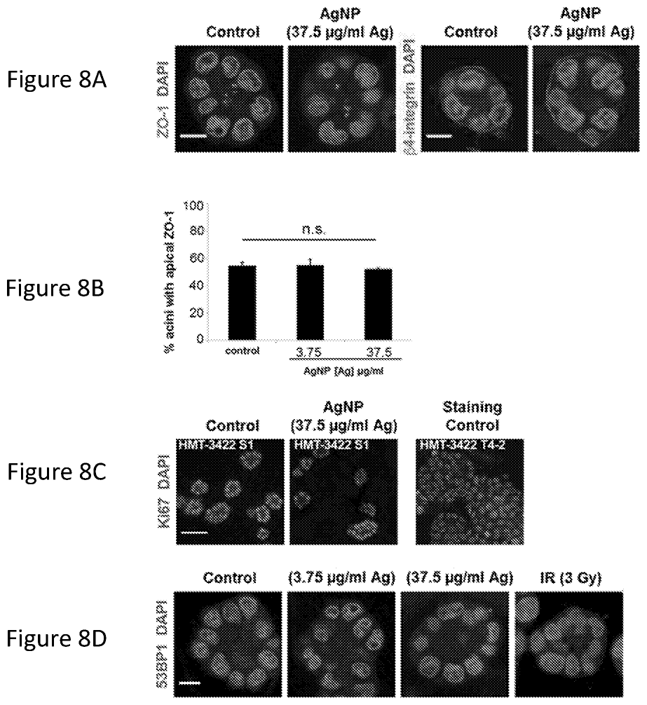

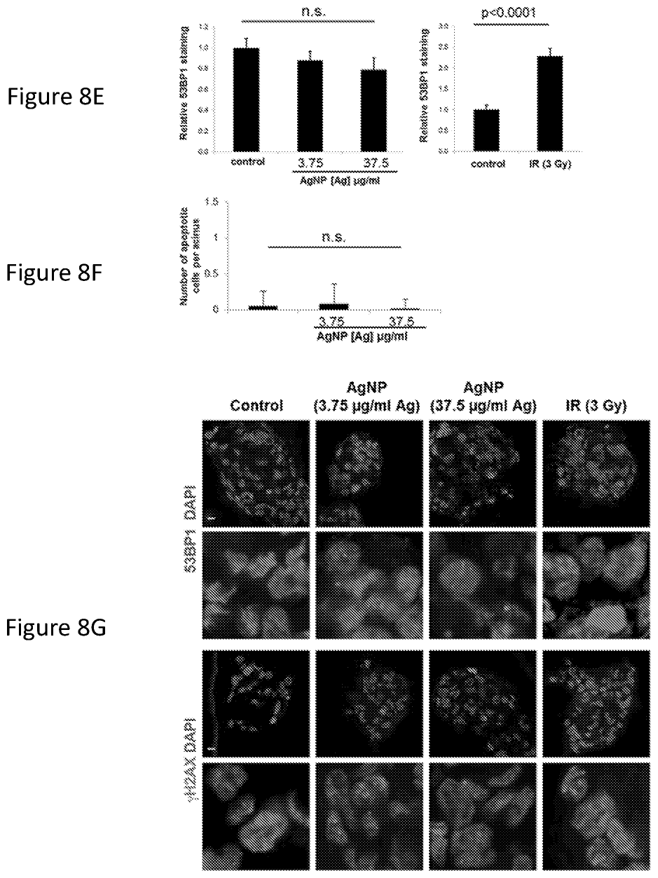

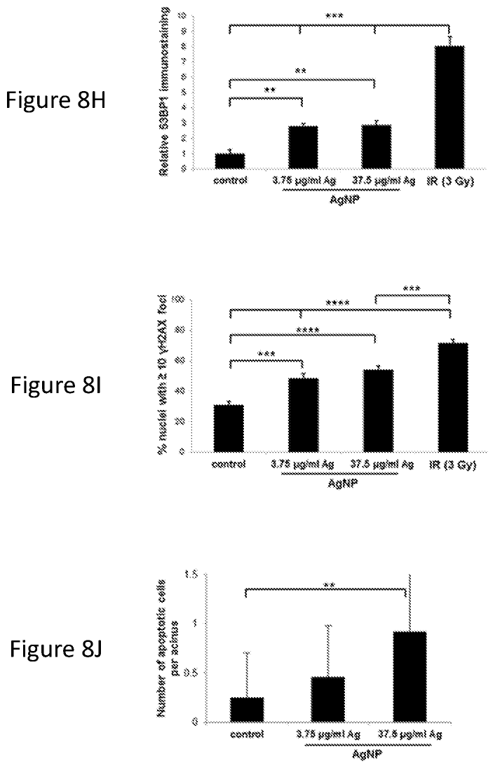

Loss of polarity is linked with breast cancer initiation. Therefore, it is key that candidate therapeutic agents do not disturb this epithelial characteristic. To assess if AgNPs affect the normal tissue architecture, 3D cultures of non-neoplastic S1 mammary epithelial cells (S1 cells) were exposed to 25 nm AgNPs (3.75 or 37.5 μg/ml of silver metal) for 48 h. S1 acini treated with AgNPs retained their characteristic single-layer spherical organization, with no multilayering, nor detectable disorganization as observed with DAPI staining, AgNP exposure did not disrupt apical localization of the TJ marker ZO-1 or basal localization of (34 integrins (FIG. 8A and B). As indicated by a lack of Ki67 staining, the AgNP treatment did not induce proliferation of S1 cells, which growth-arrest during acinar differentiation (FIG. 8C), nor did it induce detectable levels of DNA damage by 53BP1 staining (FIG. 8D and E). Treatment of S1 cells with ionizing radiation (IR) was used to validate the detection of DNA damage. Scoring pycnotic and karyorrhectic nuclei in S1 acini revealed no increase in apoptosis for AgNP-treated cells (FIG. 8F). Taken together, the results showed that AgNPs do not compromise cell homeostasis in a model of the normal mammary gland, which was consistent with the observation that AgNPs were not cytotoxic to non-malignant breast epithelial cells cultured as monolayers.

To determine if the efficacy of AgNPs towards CLBC cells is retained in a physiologically relevant context, MDA-MB-231 cells were grown in 3D Matrigel culture to produce tumor nodules. The tumor nodules were then exposed to 25 nm AgNPs, using the same dosages as for S1 acini. Both AgNP concentrations induced a significant increase in 53BP1 and γH2AX DNA repair foci in MDA-MB-231 cells compared to control, indicating DNA damage induction by AgNPs in this cell line (FIG. 8G-I). Scoring pycnotic and karyorrhectic nuclei in MDA-MB-231 tumor nodules revealed increased apoptosis in AgNP-treated cells (FIG. 8J). Collectively, the results suggested that AgNPs selectively damaged CLBC cells without compromising the normal breast epithelium.

### An Inverse Correlation Between ZEB1 and ESRP1 Expression Predict Sensitivity to AgNP Treatment in Breast Cancer

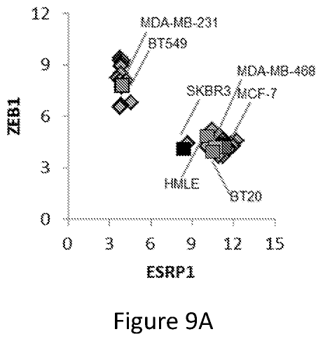

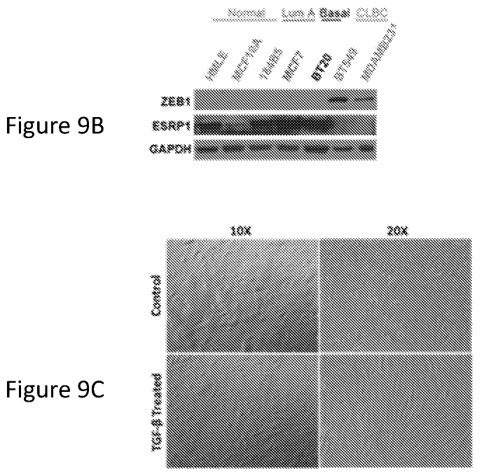

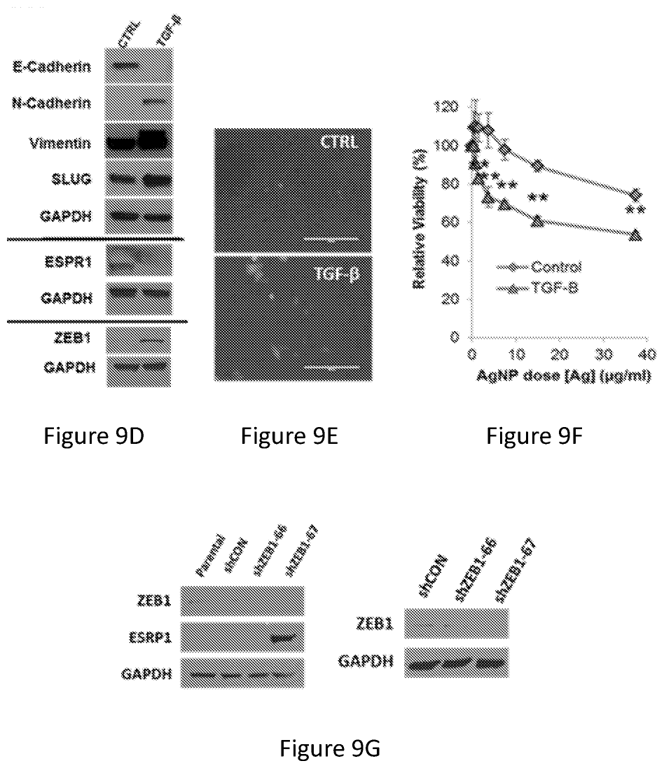

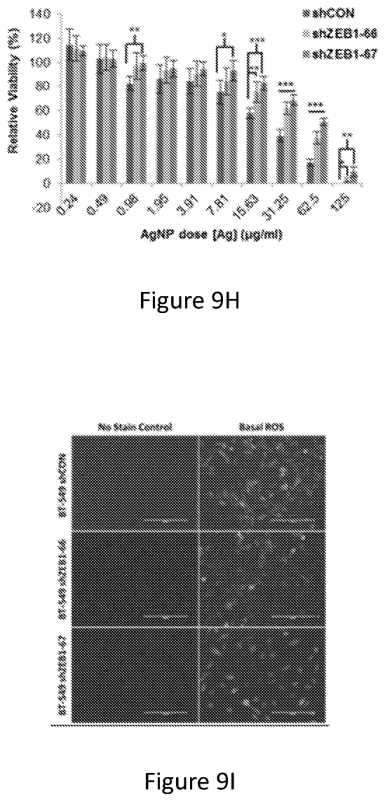

CLBC is characterized by a mesenchymal signature that also identifies a poor prognosis population of patients. ZEB1 has been identified as a transcriptional regulator of EMT, which can also stratify breast cancer patients into good and poor prognosis groups. ZEB1 represses ESRP1 preventing alternative splicing of CD44. This results in the predominance of the standard isoform of CD44 (CD44s) and thus, stem-like and mesenchymal cancer cells. Therefore, mRNA expression data for ZEB1 and ESRP1 obtained from the Broad Institute database were analyzed for breast cancer cell lines (FIG. 9A). Cell lines which were most sensitive to AgNP treatment expressed high levels of ZEB1 and low levels of ESRP1. This correlated with the cell lines of the CLBC subtype, which are also characterized by mesenchymal and stem-like signatures. Cell lines that were insensitive to AgNP treatment expressed low levels of ZEB1 and high levels of ESRP1 and corresponded with the more epithelial breast cancers. This biomarker pair was sufficient to divide the TNBC cell lines into CLBC and basal-like breast cancers. ESRP1 and ZEB1 expression was further confirmed in a panel of breast cancer and non-cancerous breast cell lines via western blot (FIG. 9B).

Because mesenchymal breast cancers were the most sensitive to AgNP treatment, transforming growth factor-β (TGF-β), which has been shown to induce EMT in a ZEB1 dependent manner, was used to generate MCF-10A non-cancerous breast cells that were more mesenchymal in phenotype. After a 6 day treatment with TGF-β, MCF-10A cells changed from a cobblestone-like morphology to an elongated, spindle-like morphology consistent with cells which had undergone EMT (FIG. 9C). Immunoblotting for markers of EMT were performed on control and TGF-β treated cells. TGF-β treated cells showed a decrease in epithelial markers, E-Cadherin and ESRP1, and an increase in mesenchymal markers, N-Cadherin, vimentin, slug and ZEB1 (FIG. 9D). Staining with the ROS activated fluorescent probe, H₂DCF-DA, showed that TGF-β treated cells exhibited higher basal ROS compared to the control cells which is consistent with cells that have undergone EMT (FIG. 9E). Control and TGF-β treated cells were exposed to increasing doses of 25 nm AgNPs for 48 h and viability was assessed via MTT assay. Cells which had undergone EMT via TGF-β treatment were significantly more sensitive to AgNP treatment compared to the control cells (FIG. 9F). This data further verifies that breast cancer cells with a mesenchymal phenotype are more sensitive to AgNP treatment when compared to breast cancer cells that are more epithelial.

To explore the role of ZEB1 in AgNP-mediated cytotoxicity, BT-549 CLBC cells, which express high levels of ZEB1 (FIG. 9A and B), were transfected with control or ZEB1 shRNAs, and knockdown was confirmed via western blot (FIG. 9G). Control and ZEB1 knockdown cells were exposed to 25 nm AgNPs for 72 h and viability was assessed via MTT assay. Knockdown of ZEB1 decreased sensitivity to AgNPs (FIG. 9H) and decreased basal ROS levels (FIG. 9I). Interestingly, when ZEB1 expression was decreased a marked increase in ESRP1 again confirmed an MET-type event. This data suggests that induction of a more epithelial phenotype via knockdown of ZEB1 decreases AgNP cytotoxicity via decreased ROS production and thus, decreased degradation of AgNPs into Ag⁺.

As understood by one of ordinary skill in the art, quantification of ZEB1 expression, ESRP1 expression, and/or ROS levels, as described herein, can be performed by one or more well-known assays for quantifying molecular molecules. Assays known for quantifying molecular molecules can include, but are not limited to, Western Blot, Northern Blot, Southern Blot, immunostaining, immunohistochemistry, PCR, qPCR, mass-spec, RT-PCR, immunofluorescence, flow cytometry, microarray, fluorescent probes, tagged probes, enzyme assay or function assay, or any molecular assay non inconsistent with the goal of the disclosure.

### Ovarian Cancer Cell Lines That Are Sensitive to AgNP treatment Can Be Identified Via ZEB1^{high}/ESRP1^{low} Biomarker Pair

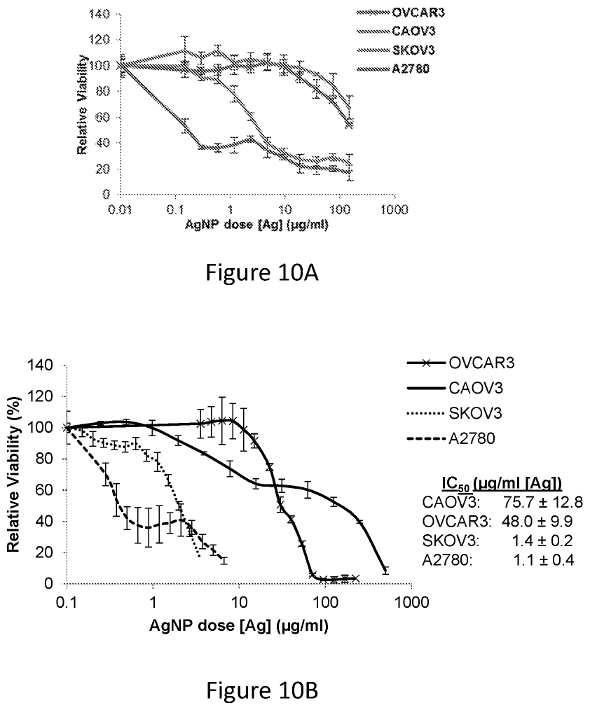

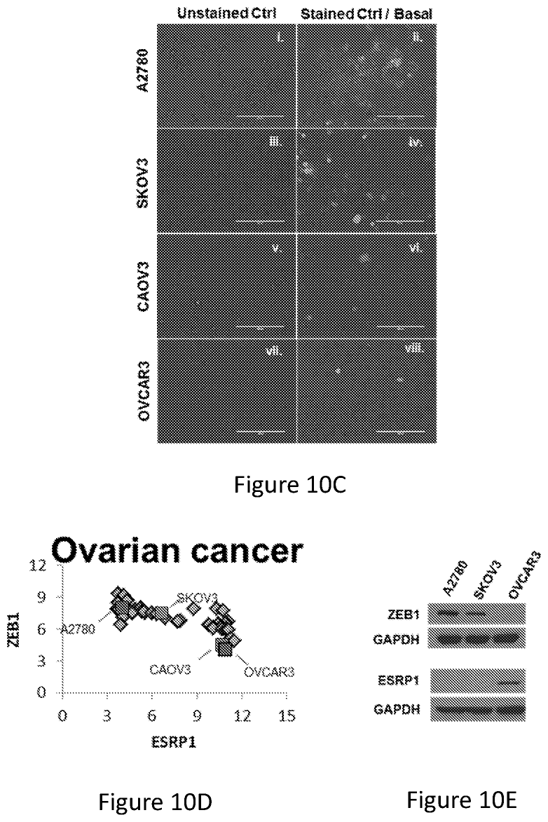

Ovarian cancer cell lines have heterogenous responses to AgNP treatment. In an expanded panel of ovarian cancer cell lines, cells were exposed to AgNPs for 48 and 72 h and viability was assessed via MTT assay. SKOV3 and A2780 cell lines were extremely sensitive to AgNP treatment, whereas OVCAR3 and CAOV3 cell lines were relatively insensitive (FIG. 10A and B). Basal levels of ROS were analyzed utilizing the ROS activated fluorescent probe, H₂DCF-DA. Consistent with results obtained for breast cell lines in FIG. 5, AgNP sensitive ovarian cancer cell lines, SKOV3 and A2780, possessed high basal levels of ROS, whereas the insensitive ovarian cancer cell lines, OVCAR3 and CAOV3, had low basal levels of ROS

(FIG. 10C). Data from the Broad Institute regarding ESRP1 and ZEB1 mRNA expression stratified the ovarian cancer cell lines tested into AgNP sensitive and insensitive subsets, where sensitive cell lines expressed high levels of ZEB1 and low levels of ESRP1, and the converse was true for insensitive cell lines (FIG. 10D). ESRP1 and ZEB1 expression in a subset of cell lines tested was confirmed via western blot (FIG. 10E).

### ESRP1 and ZEB1 Serve as Biomarkers for AgNP Sensitivity in Lung, Colorectal, and Prostate Cancer

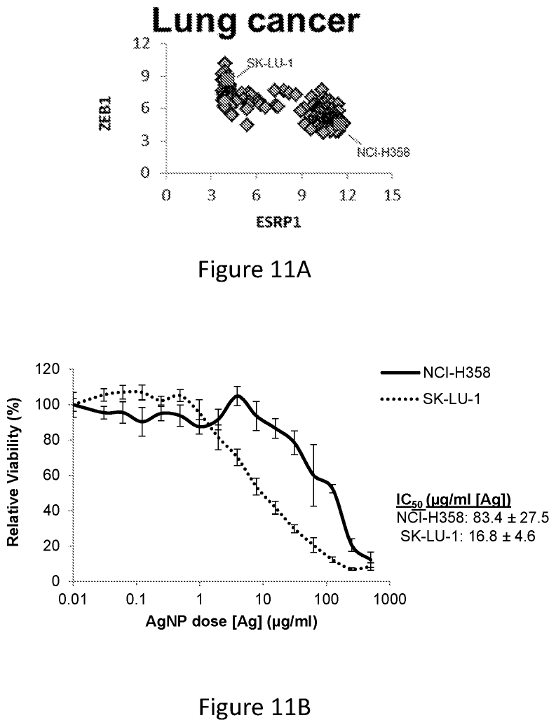

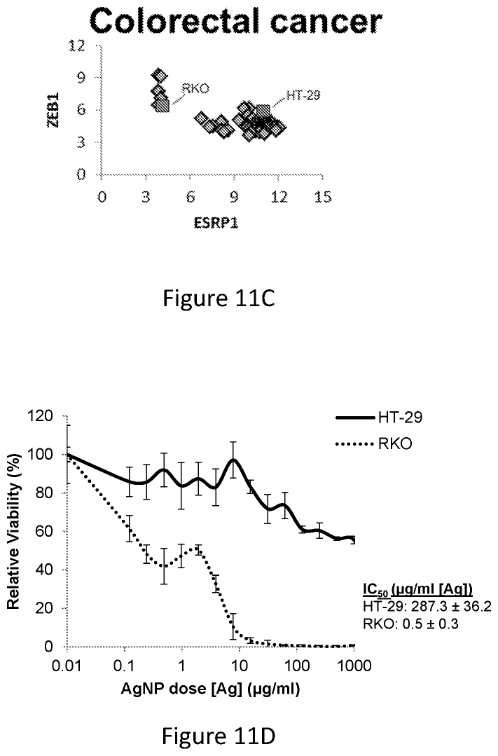

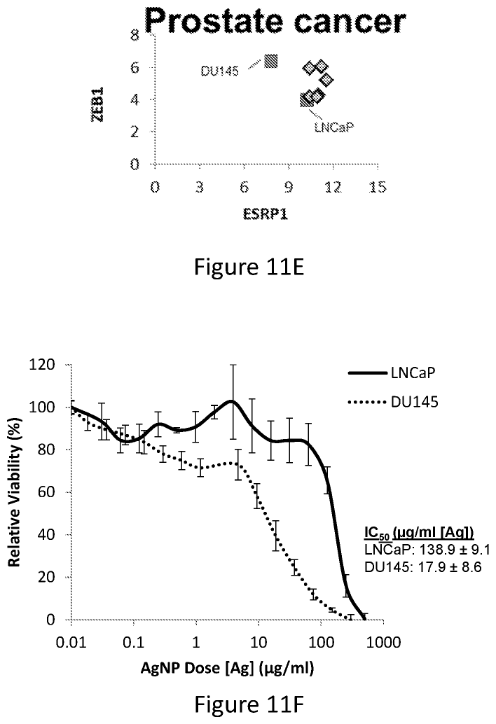

Based upon ESRP1 and ZEB1 expression in breast and ovarian cancer cell lines, other cancers that would benefit from AgNP treatment were sought based upon the biomarkers. As shown in FIG. 9A and FIG. 10D, cell lines with high expression of ZEB1 and low expression of ESRP1 were sensitive to AgNP treatment. Therefore, data from the Broad Institute was utilized to identify lung (FIG. 11A and B), colorectal (FIG. 11C and D), and prostate (FIG. 11E and F) cancer cell lines that would benefit from AgNP treatment, as well as those that would not benefit from AgNP treatment. Matched pairs of each cancer type were chosen which were either ZEB1^{high}/ESRP1^{low} (AgNP sensitive) or ZEB1^{low}/ESRP1^{high} (AgNP insensitive), and the cell lines were exposed to increasing doses of AgNPs for 72 h. Viability was assessed by MTT assay and IC₅₀ doses were calculated. As predicted, based upon our biomarker pair, SK-LU-1 lung cancer cells were sensitive to AgNP treatment with an IC₅₀ of 16.8 μg/ml Ag, whereas the NCI-H358 lung cancer cells were insensitive to AgNP treatment with an IC₅₀ of 83.4 μg/ml Ag (FIG. 11B). In the colorectal cancer cell lines tested, ZEB1^{high}/ESRP1^{low} RKO cells were extremely sensitive to AgNP treatment with an IC₅₀ of 0.5 μg/ml Ag, whereas the ZEB1^{low}/ESRP1^{high} HT29 cells were insensitive with an IC₅₀ of 287.3 μg/ml Ag (FIG. 11D). Lastly, in prostate cancer cell lines, the ZEB1^{high}/ESRP1^{low} DU145 cells were sensitive to AgNP treatment with an IC₅₀ of 17.9 μg/ml Ag, whereas the LNCaP, ZEB1^{low}/ESRP1^{high}, cells were insensitive to AgNP treatment with an IC₅₀ of 135.9 μg/ml Ag (FIG. 11F). These data suggest that the biomarker pair is sufficient to reliably identify cancers that will benefit the greatest from AgNP therapy.

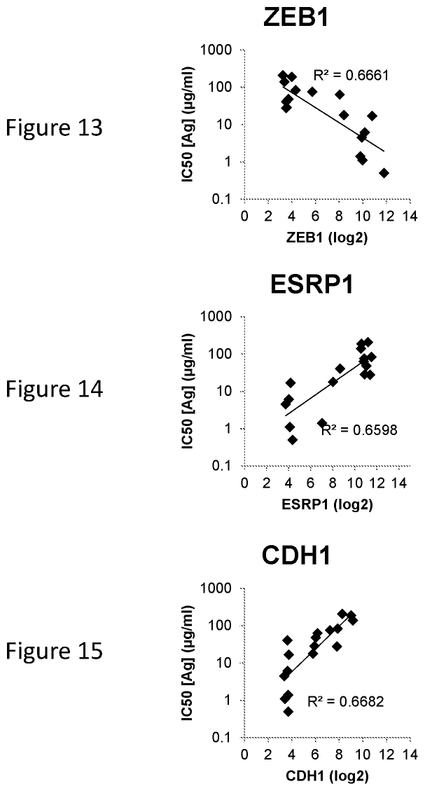

AgNPs are currently used for human medicine based on their antifouling, antibacterial and wound-healing properties. Little was known about the selectivity of AgNPs for specific cancer subtypes nor had anyone been able to successfully treat solid tumors using systemically delivered AgNPs. Several embodiments described herein demonstrate the use of AgNPs as a therapeutic agent for systemic treatment of CLBC tumors in mice and supports in vivo findings with in vitro evidence showing that AgNPs are highly cytotoxic to CLBC cells at doses that do not induce cytotoxicity or otherwise disrupt the homeostasis of non-cancerous breast epithelia. CLBC-selective cytotoxic properties of AgNPs are independent of particle size, shape or capping agent. CLBC-specific cytotoxicity of AgNPs is not shared by ionic silver and is therefore, one of the first examples of a “new to nano” cytotoxic property. Mechanistically, CLBC cells possess high basal levels of ROS, which induce degradation of AgNPs into Ag ion causing DNA damage, ER stress, redox imbalance, and apoptotic cell death without causing similar damage or cell death in non-cancerous breast cells. Non-cancerous breast cells possess low levels of basal ROS which decreases the likelihood of AgNP degradation into Ag ion, preventing cell damage and apoptosis. Furthermore, AgNPs do not disrupt the architecture of non-neoplastic breast epithelial cells grown in 3D cell culture, nor do they cause DNA damage, or induce apoptosis in these cells. In contrast, AgNPs, at doses that were non-toxic to non-neoplastic breast epithelial cells, cause extensive DNA damage and apoptosis in CLBC cells grown in 3D culture. Most importantly, intravenously injected AgNPs are effective for the treatment of CLBC xenografts in mice without acute off-target toxicity. These data support the possibility that AgNPs may be useful for treatment of metastatic breast cancer. When data from all cancer types is pooled, it can be seen that expression of ZEB1 is negatively correlated with the IC₅₀ of AgNP treatment (FIG. 13). Conversely, the transcriptionally repressed targets of ZEB1, ESRP1 and CDH1, are positively correlated with the IC50 of AgNP treatment (FIGS. 14 and 15). Of note, some embodiments described herein provide biomarkers and combinations of biomarkers, ZEB1^{high}/ESRP1^{low}/CDH1^{low} that each individually or in any combination correspond with cancers possessing a more mesenchymal phenotype, which successfully distinguished cell lines across a variety of cancers that are sensitive to AgNPs (FIGS. 13, 14, 15). The corresponding biomarkers identified for sensitivity to AgNP treatment have the capability to identify candidate patients with aggressive, mesenchymal cancers that currently have limited treatment options who would benefit from AgNP treatment.

HSF1 is a master regulator of cellular proteotoxic stress; it guards against proteomic stability and enables stress adaptation. HSF1 is essential for transcription of chaperones to maintain proteomic stability and regulate non-HSP genes involved in essential cell processes.

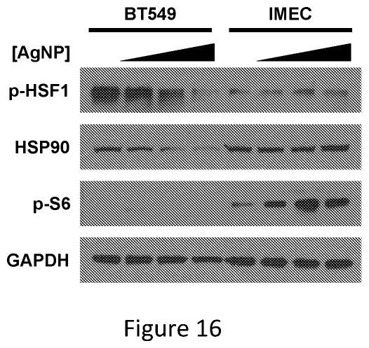

Impaired HSF1 function increases proteotoxic stress and decreases survival adaptations. Notably, AgNPs inhibited the phosphorylation (activation) of HSF-1 in BT549 claudin-low breast cancer cells at doses that did not affect HSF-1 activation in immortalized mammary epithelial cells (IMECs) (FIG. 16). Furthermore, expression of HSP90, a target of HSF1, decreases with AgNP treatment in a dose-dependent manner. In contrast, HSP90 expression in IMECs is not affected by AgNP treatment.

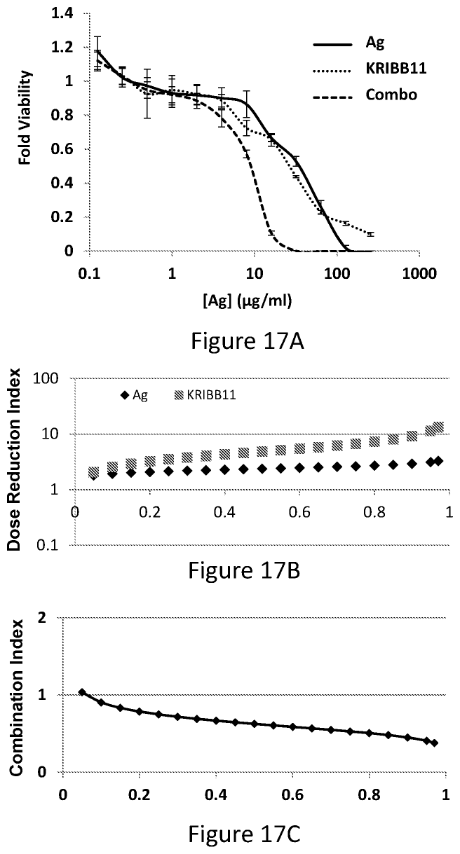

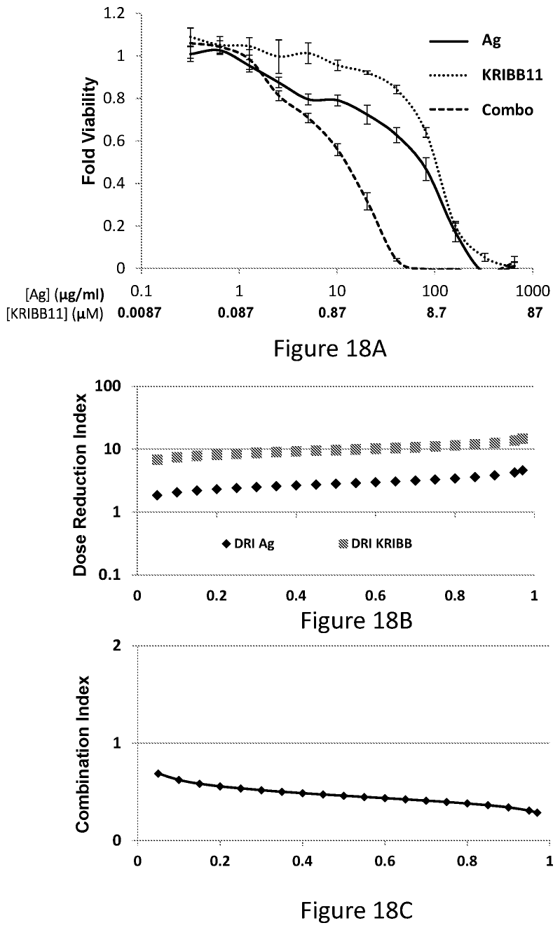

KRIBB11 is a small molecule inhibitor of HSF1. BT549 CLBC cells (FIG. 17) and BT20 basal-like cancer cells (FIG. 18) were treated with AgNPs and KRIBB11, independently and in combination. Each treatment was assessed for its dose reduction index and combination index. Combination treatment of both AgNPs and HSF1 inhibition showed synergistic effects in both breast cancer cell types. Synergism, as understood by a skilled artisan, occurs when the combined effect observed is significantly greater than the expected (additive) effect of each treatment. Although AgNP treatment and HSF1 treatment synergized, AgNP treatment and inhibition of HSP90, a molecular target downstream of HSF1, did not synergize when BT549 cells were exposed to the combination. Nevertheless, combination of AgNP treatment and HSP90 inhibition still resulted in an additive effect. It should be understood by a skilled artisan that synergism and addition are quantifiably distinct treatment outcomes.

Methods described herein can include quantifying ZEB1 alone or in combination with any one or more of ESRP1, CDH1, and/or HSF1. Whereas ZEB1, ESRP1, and CDH1 are each quantified according to their expression, HSF1 can be quantified by its expression and/or activation, wherein HSF1 activation is determined by quantifying pHSF1 expression. For example, a method can include quantification of the following combinations of biomarkers: ZEB1, ZEB1/ESRP1, ZEB1/CDH1, ZEB1/HSF1, ZEB1/pHSF1, ZEB1/ESRP1/CDH1, ZEB1/ESRP/HSF1, ZEB1/ESRP/pHSF1, ZEB1/CDH1/HSF1, ZEB1/CDH1/pHSF1, ZEB1/HSF1/pHSF1, ZEB 1/ESRP1/CDH1/HSF1, ZEB1/ESRP1/CDH1/pHSF1, ZEB1/ESRP1/HSF1/pHSF1, ZEB1/CDH1/HSF1/pHSP1, and/or ZEB1/ESRP1/CDH1/HSF1/pHSF1. Furthermore, expression of each biomarker can then be compared with a threshold of the same biomarker above or below which a cancerous cell population responds to silver nanoparticles. Thus, in some embodiments, methods described herein can comprise quantifying the expression of ZEB1 alone or in combination with any one or more of ESRP1 expression, CDH1 expression, HSF1 expression, and/or HSF1 activation.

The outcomes of NP toxicity testing are challenging because factors that affect physicochemical features such as particle size, -potential, and reactivity can also influence colloidal properties which in turn affect solution dynamics, cell uptake, intracellular trafficking, exposed dose and cytotoxicity. The difficulty of identifying which factor contributes to a particular toxicity profile is daunting, and likely plays a role in the lack of reproducibility of many of the studies that attempt to do so. The toxicity of identical AgNPs on different breast cancer and non-cancerous cell lines was assessed to identify unique aspects of the AgNP toxicity profile that are dependent upon the underlying biology of the cell target. Using this approach, a novel aspect of AgNP cytotoxicity was identified: CLBC cells are extremely sensitive to AgNP exposure. Particles used in earlier studies tended to aggregate in physiological solution. If particles aggregate under these conditions, it is likely they will also aggregate in blood and be rapidly cleared by phagocytes, become entrapped in lung capillary beds, or fail to diffuse through extracellular matrix, all of which will prevent them from reaching their tumor target when injected systemically. Additionally, for clinical development of AgNP-based therapeutics, it will be necessary to clearly define the specific physicochemical features of the nanoparticles that will be used. Therefore, it is important that the AgNPs be monodisperse with regard to size, shape, ζ-potential and that they did not aggregate under physiologic pH and ionic strength.

Notably, the CLBC-selective properties were dependent upon the use of intact AgNPs, but were conserved regardless of changes in AgNP size, shape, or capping agent. This means that there will be great versatility to tailor size, shape, and surface properties to optimize the tumor targeting and body clearance of AgNPs for future in vivo applications without loss of CLBC selective cytotoxicity. The PVP-coating used for the majority of the AgNPs in this study may offer an advantage over other polymer coatings such as PEG. Specifically, repeated injection of PEG-coated nanoparticles may induce an accelerated blood clearance in which the blood circulation time decreases for subsequent injections of PEG-coated nanoparticles, a phenomenon not found for repeated administration of PVP-coated nanoparticles. Additionally, the negative potential of the PVP-coated AgNPs may be favorable for in vivo use because positively charged AgNPs are rapidly cleared from the circulation and induce liver toxicity in mice.

In vitro testing of AgNP cytotoxicity identified that CLBC cells were significantly more sensitive to AgNP treatment after 48 h and 72 h exposure. A 6 h pulse with AgNPs in MDA-MB-231 CLBC cells was sufficient to induce cytotoxic effects after 72 h indicating that constant exposure to AgNPs is not critical for cytotoxicity, but because AgNPs act as pleiotropic stressors, the additional time is necessary for cell death to become apparent. Non-cancerous breast cell lines were relatively insensitive to AgNP treatment at both exposure times, whereas basal-like, HER2 overexpressing, and luminal A breast cancer cell lines were moderately sensitive. The SKBR3, HER-2 overexpressing, cell line exhibited a 2-step cytotoxicity curve, where some cells were sensitive to AgNP treatment at low doses, but the overall IC₅₀ for these cell lines was almost 10 times higher than well classified CLBC cell lines, MDA-MB-231 and BT-549. Additionally, the SKBR3 cell line exhibited high basal levels of ROS and expressed high protein levels of ZEB1 and low protein levels of ESRP1, which did not correlate with the mRNA expression data obtained from the Broad Institute. This deviation from other moderately sensitive cell lines suggests that the SKBR3 cell line contains a mixed population of cells, which was observed in a previous study where a “side population” possessing CSCs was identified in the SKBR3 cell line. Because CLBC cells are characterized by stem-cell like properties, it is highly conceivable that this “side population” of SKBR3 cells are the ZEB1^{High} expressing stem-like cells that are sensitive to AgNPs. For breast cancers with mixed populations and the more epithelial cancers, the radiosensitizing properties of AgNPs previously demonstrated may prove beneficial for therapy.

Several previous studies indicate that AgNPs can induce ER stress, though none have shown that AgNPs selectively induce ER stress in CLBC cells. ER stress can activate three arms of the UPR, each of which is referred to by its initiating stress sensor, which include inositol-requiring protein 1 (IRE1) and activating transcription factor-6 (ATF-6) in addition to PERK. Under normal conditions, GRP78 is sequestered at the ER membrane by these stress sensors. However, in the presence of misfolded proteins, these complexes dissociate to initiate the UPR. There are conflicting reports on activation of the IRE1 arm by AgNPs with one study indicating its activation following AgNP exposure and another showing no change. This may be due to the fact that these studies also differed in the type of cells used to evaluate this response. Less is known about the role of the ATF-6 arm following AgNP exposure, but there are some indications that AgNPs degrade ATF-6 in some cell lines. There is a growing interest in the development of cancer treatment agents that can selectively induce ER stress, and further studies are warranted to understand this aspect of the CLBC-specific AgNP cytotoxicity discovered by our study.

Induction of autophagy may play a role in AgNP toxicity. AgNPs and their degradation products can be found by TEM in autophagic vesicles in MDA-MB-231 cells. How and why this occurs only in these cells and not in MCF-10A cells and how AgNPs might affect autophagic flux in CLBC cells versus other cell types is a subject for further investigation. As noted above, this may in part be due to the high basal levels of ROS observed in MDA-MB-231cells, which could increase the degradation of AgNPs and increase Ag+ release. High basal ROS is a property shared among CLBC cell lines and basal ROS levels correlate with AgNP sensitivity in a panel of breast cancer and non-cancerous breast cells spanning the molecular subtypes of breast cancer. The ability of a 4 h NAC pre-treatment to mitigate AgNP-induced cytotoxicity in the two most sensitive CLBC cell lines (FIG. 6C), suggests that high basal ROS levels play a leading role in AgNP cytotoxicity. SOD2 and catalase levels were assessed in a panel of breast cell lines, as the ratio of SOD2 to catalase has been implicated as a potential biomarker for cancers that may benefit from treatments that induce oxidative stress. However, SOD2 and catalase levels or the ratio between the two did not correlate with high basal levels of ROS seen in FIG. 5, or sensitivity to AgNP treatment. Therefore, while high basal ROS does correlate with AgNP and appears to play a vital role in AgNP cytotoxicity, SOD2 and catalase protein expression does not identify cell lines that will benefit from AgNP treatment. Future studies examining the influence of intracellular ROS on AgNP degradation and specific protein targets of AgNPs in CLBC could further identify vulnerabilities that could be exploited for therapy.

Results indicate that AgNP treatment increases the percentage of MDA-MB-231 cells in S and G2/M phases relative to G0/G1. This is particularly notable because ER stress and the UPR are expected to induce arrest in G0/G1. Effects of AgNPs on the cell cycle are most apparent in the CLBC cells, which is consistent with data showing that AgNPs induce DNA damage in these cells but not in non-neoplastic breast epithelia. The slowing of progression through S-phase after AgNP exposure may occur as cells attempt to repair damaged DNA. It is possible that at later time points AgNP-induced ER stress would cause G1 arrest, but initiation of apoptosis interferes with such analysis. Furthermore, there is significant crosstalk between ER stress and DNA damage response pathways, and it may be difficult to specifically identify effects due solely to each type of damage. Nonetheless, it is clear that AgNPs exert their CLBC-selective cytotoxicity through a pleotropic combination of stresses.

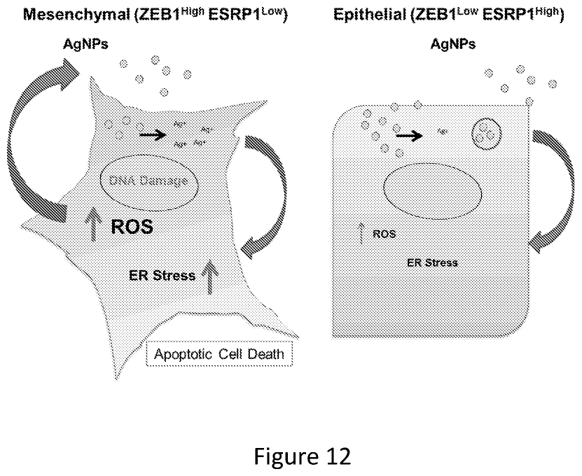

Results identified cell lines with high expression of ZEB1 and low expression of ESRP1 as AgNP sensitive cell lines in breast, ovarian, lung, colorectal, and prostate cancer. ESRP1 is necessary for the splicing of CD44 which leads to an increase of CD44v and a decrease in the standard isoform, CD44s. When cells undergo EMT via extracellular factors such as HGF or TGF-β, the CD44s isoform predominates due to ZEB1 repression of ESRP1. This forms a feedback loop where CD44s activates ZEB1 for continued suppression of ESRP1 and thus, maintenance of the mesenchymal phenotype. TGF-β mediated EMT induction in MCF-10A non-cancerous cell line (FIG. 9C-F) further demonstrated that more mesenchymal breast cancers have higher basal levels of ROS and are more sensitive to AgNPs. Furthermore, knockdown of ZEB1 significantly decreased AgNP sensitivity in BT-549 CLBC cells, indicating that ZEB1 plays a functional role in AgNP-induced cytotoxicity. Additional studies are necessary to identify if a similar mechanism of action for AgNP-mediated cytotoxicity, outlined in FIG. 12, holds true in lung, colorectal, and prostate cancers. However, regardless of mechanistic differences that may exist between cancers, the ZEB1^{High}/ESRP1^{Low} biomarker pair is sufficient to identify AgNP sensitive cancers. Because antibodies against these proteins are readily available, it is feasible that this biomarker pair could be quickly translated to the clinic for identification of patients that will benefit the greatest from AgNP therapy.

Detailed in vivo toxicological studies were not performed; however, the 24 h biodistribution study showed that AgNPs accumulated in the liver with lower levels detected in the lungs, spleen, and kidneys. Low levels of Ag were detected in the urine, which corresponds with the low levels of Ag detected in the kidneys. This suggests that the AgNPs are not cleared by the kidneys, which is consistent with knowledge that NPs larger than 10 nm will not be cleared by the kidneys, but are more likely to be cleared by the MPS. Comprehensive studies in rodents have been performed using PVP-stabilized AgNPs produced with similar characteristics to the present AgNPs. For example, after exposing rats to a 28 day repeated intravenous AgNP dose of 6 mg/kg, no dose limiting toxicity was observed, though transient effects on liver and immune cell function were noted. Similarly, there were no AgNP-associated, dose limiting toxicities observed in rats during 28 day and 90 day inhalation studies or after a 28 day oral toxicity study in which rats were given AgNP doses up to 1000 mg/kg per day. A gradual decrease in silver content from liver, spleen and other organs was detected during a two month follow-up study in rats orally dosed with AgNPs (90 mg/kg) daily for one month. Proteomic analysis of plasma proteins coating the AgNP surface indicate binding of complement or pro-coagulant plasma proteins is low, which is in agreement with studies showing that AgNPs do not affect platelet aggregation, the coagulation process, or complement activation. In addition to the results reported here, the safety in rodents of systemically injected AgNPs for use in cancer imaging, vaccines and cancer immunotherapy has been demonstrated and further supports the potential for clinical translation of AgNPs. Noble metal-based, engineered nanomaterials possess unique optical, electrical, and thermal properties that allow for multimodal activity without the need for additional engineering. For clinical translation to become a reality, the potential risk-benefit balance must be resolved for these materials. The research presented here suggests the potential for a paradigm change in the development of these types of nanomaterials for cancer applications. According to this new paradigm, nanomaterials should be deemed most suitable for further development toward specific clinical indications only if their properties confer a unique advantage in the context of the underlying biology of that application. In a similar vein, the present AgNP formulation shows efficacy against CLBC following intravenous injection in tumor bearing mice. The present nanoparticles consist of only two components: silver and a dense stabilizing layer of PVP, a biocompatible polymer considered generally safe by the United States Food and Drug Administration (FDA). The simplicity of these two component NPs, when added to existing facile, scalable production capabilities, makes them most attractive for further development. The present study suggests a window exists for the safe use of AgNPs for treatment of CLBC and lays the foundation for the development of AgNPs, and therefore offers the possibility of a major benefit to this poor prognosis patient population. Additionally, similar vulnerabilities in ovarian cancer are herein identified indicating in vivo studies are warranted. In the future, it will be possible to build upon the unique interaction of AgNPs with CLBC to determine which properties of the nanomaterial or the cancer target are important to retain or enhance this CLBC selective response. Given the extensive use of AgNPs in current medical practice, the lack of treatment options for women with recurrent CLBC and the identification of the ZEB1/ESRP1 biomarker pair that can identify patients most likely to benefit from AgNP therapy, human clinical trials involving AgNPs would be highly conceivable.

Many modifications and other embodiments of the subject matter will come to mind to one skilled in the art to which the subject matter pertains having the benefits of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

Various implementations of silver nanoparticles and methods of using the same have been described, and exemplary embodiments are described below in fulfillment of various objectives of the present disclosure. It should be recognized that these implementations are merely illustrative of the principles of the present disclosure. Numerous modifications and adaptations thereof will be readily apparent to those skilled in the art without departing from the spirit and scope of the present disclosure. For example, individual steps of methods described herein can be carried out in any manner not inconsistent with the objectives of the present disclosure, and various configurations or adaptations of silver nanoparticles described herein may be used.

### EXAMPLE 1

### Physicochemical Characterization of AgNPs and Effects of AgNP Size and Particle Formulation on CLBC-Selective Cytotoxicity

(FIG. 1A) TEM images were taken of AgNPs (5, 25, 50, 75 nm in diameter) obtained from nanoComposix, Inc. (FIG. 1B) The hydrodynamic diameter of 5 nm AgNPs were analyzed by DLS and 25, 50, and 75 nm AgNPs were analyzed by NTA. (FIG. 1C) The hydrodynamic diameter of 25 nm AgNPs dispersed in H₂O, PBS, or DMEM supplemented with 10% FBS was analyzed over time. MDA-MB-231 and MCF-10A cells were exposed to (FIG. 1D) AgNPs (5, 25, 50, or 75 nm in diameter), (FIG. 1E) AgNPs (25 nm) or silver nitrate (as a source of Ag+), (FIG. 1F) 20 nm gold nanoparticles (AuNP) for 48 h, or (FIG. 1G) silica-shelled AgNPs (Ag@SiNP). Viability was assessed by MTT Assay. Significant differences are indicated (ANOVA; T-Test; **p<0.01).

### EXAMPLE 2

### Cytotoxicity of AgNPs in a Panel of Breast Cancer and Non-Cancerous Cell Lines

A panel of breast cells lines of various cancer subtypes listed in Table II were treated with AgNPs (25 nm) for (FIG. 2A) 48 h or (FIG. 2B) 72 h. Viability was assessed by MTT Assay. Significant differences are indicated (Tukey Test; ***p<0.001).

Molecular subtype classification of breast cell lines treated for 72 h with 25 nm AgNPs and viability was assessed by MTT assay. IC₅₀ calculations were performed using the prism software. IC₅₀ in Table II are shown in total NP (AgNP) concentration and silver (Ag) content. At least 3 independent experiments were conducted for each cell line.

### EXAMPLE 3

### Systemic Delivery of AgNPs Slows Growth of CLBC Tumors In Vivo

Tumor bearing mice (9 per group) were intravenously injected with PBS or AgNPs (6 mg/kg; 3× per wk; 10 wks). (FIG. 3A) Tumor growth was quantified over time by caliper measurements and the start (first Rx) and end (final Rx) of treatment is indicated. Significant differences in tumor size are indicated by asterisks “*” for each time point (Mann-Whitney). (FIG. 3B) Animal weight was assessed over time and indicates no difference between AgNP and control groups. (FIG. 3C) Survival of treated mice is plotted by Kaplan-Meyer analysis. Due to tumor growth in excess of the severity limit of the protocol (>1000 mm) ⅔ of PBS treated mice were euthanized prior to the end of the study at 100 days. No tumors in AgNP treated mice reached the tumor size limit and all mice survived until the completion of the study at 100 days. (FIG. 3D) Images are provided showing tumor necrosis in a mouse treated intravenously with AgNPs or a large tumor mass in a PBS mouse at the study end point. (FIG. 3E) Silver content (parts per trillion; ppt) in tumors of mice reaching the study endpoint was quantified by ICP-MS. (FIG. 3F-FIG. 3H) Biodistribution of AgNPs (intravenous AgNP treatment of 6 mg/kg) after 24 h was quantified in (FIG. 3F) Organs (FIG. 3H) and Urine. Biodistribution of AgNPs (lintravenous AgNP treatment of 6 mg/kg) after 24 h was quantified in (FIG. 3G) blood over the course of 24 hrs.

### EXAMPLE 4

### AgNPs Exhibit Differential Internalization, Intracellular Trafficking, and Degradation in CLBC and Non-Cancerous Breast Cells

MDA-MB-231 and MCF-10A cells were exposed to AgNPs (25 nm) for 6 or 24 h. (FIG. 4A) The amount of Ag taken up by the cells was quantified by ICP-MS. Separately, cells were pulsed with AgNPs for 1 h to allow internalization and then chased for a further 6 h to allow time for intracellular trafficking. Internalized AgNPs were visualized by TEM (30000× magnification). The images show AgNPs in MCF-10A cells after 1 h (FIG. 4B; panels i and ii) and 6 h (FIG. 4B; panels iii and iv), or in MDA-MB-231 cells after 1 h (FIG. 4C; panels v and vi) and 6 h (FIG. 4C; panels vii and viii). AgNPs in MDA-MB-231 cells appear to be degraded compared to AgNPs in MCF-10A cells. Early endosomes containing AgNPs are indicated by white arrows, and lysosomes or amphisomes containing AgNPs are indicated by the black arrows. Organelles and vesicles are identified in the images: AM (amphisome); EE (early endosome); Ly (lysosome); Mt (mitochondria); N (nucleus).

### EXAMPLE 5

### Basal Levels of ROS and ROS Levels Following Treatment With AgNPs Differ Across Breast Cancer Subtypes and Non-Cancerous Breast Cell Lines and Influences Release Of Ag+ From AgNPs

The H₂O₂ mediated dissolution of 25 nm AgNPs to release Ag+, indicated by a decrease in plasmon resonance absorption, was monitored by UV/Vis spectroscopy. (FIG. 5A) Dissolution of AgNPs (as indicated by a decrease in the relative height of the plasmon resonance absorbance peak) was quantified over time at pH 7 or pH 4 in the presence or absence of 100 μM H₂O₂. The data are presented as the mean of three independent experiments±standard deviation. Significant differences are indicated (***p<0.001; 2 way ANOVA with post-hoc Tukey Test). (FIG. 5B) CLBC (MDA-MB-231, BT-549, SUM-159), luminal A (MCF-7), HER2+ (SKBR3), basal-like (MDA-MB-468 and BT-20) breast cancer cells and non-cancerous breast cells (MCF-10A and iMEC) were seeded and allowed to attach the overnight, then treated with vehicle, NAC or AgNPs (25 nm) for 24 h. Cells were washed with PBS, incubated with PBS containing the ROS responsive dye, H2DCF-DA, and then ROS was assessed by fluorescence microscopy. Dye-free (Unstained Ctrl) controls of cells treated were used to verify the specificity of the fluorescence for ROS detection.

### EXAMPLE 6

AgNPs Induce Redox Imbalance and Cytotoxicity in CLBC, Which Can Be Mitigated Via NAC Pre-Treatment, But is Independent of SOD2 or Catalase Expression

The ratios between (FIG. 6A) reduced and oxidized glutathione (GSH/GSSH) or (FIG. 6B) reduced and oxidized nicotinamide adenine dinucleotide phosphate (NADPH/NADP+) were quantified in cell lysates following exposure of MDA-MB-231 or MCF-10A cells to AgNPs (25 nm) for 24 h. Significant differences between treatment groups are indicated (ANOVA; T-Test; *p<0.05*; **p<0.01). N.S., non-significant (ANOVA, p>0.05). (FIG. 6C) MDA-MB-231 and BT-549 cells were pre-treated with NAC or vehicle, treated for with AgNPs (25 nm) for 48 h, and viability was assessed via MTT assay. (ANOVA; T-Test; **p<0.005; p<0.0005). (FIG. 6D) SOD2 and catalase relative to GAPDH expression in a panel of breast cell lines were analyzed by western blot.

### EXAMPLE 7

AgNPs Induce ER Stress, Apoptosis, and Slow Cell Cycle Progression Through S-Phase in CLBC Cells, Without Affecting Non-Cancerous Breast Cells

MDA-MB-231, MCF-7 and MCF-10A were treated with AgNPs (25 nm) for 6 or 24 h, and then cell lysates were analyzed for markers of ER stress by western blot, as indicated. (FIG. 7A) Representative western blots show that AgNPs induce ER stress in MDA-MB-231 (CLBC) cells but not in non-TNBC MCF-7 or MCF-10A cells. Protein levels relative to the β-actin loading control were quantified by densitometry (n=5). Expression of PERK, p-eIFα/total eIFα, GRP78, and CHOP is shown relative to levels detected in untreated MCF-10A cells. Significant differences in protein levels relative to baseline are shown (T-Test; *p<0.05 as indicated in the figure). (FIG. 7B) MDA-MB-231 or MCF-10A cells were treated with AgNPs (25 nm) for 48 h. Cells were co-stained with propidium iodide and Annexin V and then evaluated by flow cytometry. The percentages of cells characterized as viable (lower-left quadrant), early apoptotic (lower-right quadrant), late-apoptotic (upper-right quadrant), and necrotic (upper-left quadrant) are shown within each quadrant. (FIG. 7C) MDA-MB-231, MCF-7 and MCF-10A were treated with AgNPs (25 nm) or vehicle (PBS) for 6 or 24 h, stained with PI and then cell cycle distribution was analyzed. The relative proportion of cells in each phase of the cell cycle (G0/G1; S; and G2M) is indicated in each panel.

### Example 8

### AgNPs Do Not Alter Normal Breast Homeostasis, But Induce DNA Damage and Apoptosis in CLBC

(FIG. 8A) Confocal images of S1 acini treated for 48 h with AgNP (25 nm) or with PBS (control) and immunostained for the tight junction marker ZO-1 or the basal marker (34 integrin are shown. Nuclei were counterstained with DAPI. (FIG. 8B) Apical ZO-1 localization in acini treated as in FIG. 8A are quantified. Mean±standard error from 3 biological replicates are shown. At least 100 structures were scored per condition for each replicate. No significant differences were detected between treatment groups (N.S.; ANOVA, p>0.05). (FIG. 8C) Confocal images of immunostained S1 acini differentiated in 3D culture for the proliferation marker Ki67 are provided. Ki67 staining was validated by parallel analysis of S1-derived T-42 breast cancer cells. (FIG. 8D) DNA damage was detected by immunostaining for 53BP1 in S1 acini treated with AgNP or PBS. Irradiation (3 Gy, IR) was used for validation. (FIG. 8E) DNA damage was then quantified. For each acinus cross-section, the average number of 53BP1 foci/nucleus in confocal images of S1 acini was quantified. The bar graph represents mean±standard error (N>20 acini from two independent biological replicates) after normalization to PBS-treated cells. No significant differences between AgNP treatment groups were detected (N.S.; ANOVA, p>0.05). However, significant differences (as indicated) in 53BP1 foci were detected between acini exposed to IR or mock-irradiated (control). (FIG. 8F) The number of apoptotic cells per acinus was estimated based on pyknosis and karyorrhexis detected with DAPI staining of S1 acini treated as in FIG. 8A. No significant differences in between treatment groups were detected (ANOVA; p>0.05; N>20 acini from two independent biological replicates). Scale bars=10 μm. (FIG. 8G) 53BP1 (green) and phosphorylated H2AX (γH2AX, red) were detected immunofluorescence as provided by confocal microscopy in MDA-MB-231 cells cultured in 3D with Matrigel. Cells were treated for 48 h with PBS (control) or PVP-coated AgNPs (25 nm). Exposure to 3 Gy of ionizing radiation (IR) served as control. Scale bars=10 μm. Zoomed images are shown in the lower panels for each stain. (FIG. 8H) For each nodule cross-section of MDA-MB-231 cells treated as in FIG. 8G, the average number of 53BP1 foci/nucleus was scored. Means±standard error are shown after normalization to control. Significant differences between treatment groups were detected as indicated (ANOVA; **p<0.01 and ***p<0.001; N≥7 nodules from two independent biological replicates). (FIG. 8I) The proportion of nuclei with at least 10 γH₂AX foci per cross-section was quantified in MDA-MB-231 cells treated as in FIG. 8G. Significant differences between treatment groups were detected as indicated (ANOVA; ***p<0.001 and ****p<0.0001; N=9 nodules from two independent biological replicates). (FIG. 8J) The number of apoptotic cells per nodule was estimated based on pyknosis and karyorrhexis detected with DAPI staining in confocal images of MDA-MB-231 tumor nodules treated as in FIG. 8G and significant differences in between treatment groups were detected as indicated (ANOVA; ** p<0.01; N=9 nodules from two independent biological replicates).

### EXAMPLE 9

### ZEB^{high}/ESRP1^{low} Expression Predicts Sensitivity to AgNP Treatment in Breast Cancer

(FIG. 9A) mRNA expression data was obtained from the Broad Institute database for ZEB1 and ESRP1 in breast cancer cell lines. ZEB1 expression was plotted against ESRP1 expression. (FIG. 9B) ZEB1 and ESRP1 protein expression was assessed via western blot in a panel of breast cell lines. MCF-10A non-cancerous breast cells were treated with vehicle or TGF-β for 6 days. (FIG. 9C) Light microscopy images of the cells were taken to show morphological differences. (FIG. 9D) Markers of EMT (E-cadherin, N-cadherin, vimentin, slug, and ESRP1) relative to GAPDH were assessed via western blot. (FIG. 9E) Cells were incubated with the ROS responsive dye, H2DCF-DA, diluted in PBS and then ROS was assessed by fluoresence microscopy. (FIG. 9F) Cells were exposed to AgNPs (25 nm) for 48 h and viability was assessed by MTT assay (ANOVA; T-Test; *p<0.05, **p<0.01, ***<0.001). BT-549

CLBC cells were transfected with control or ZEB1 shRNA. (FIG. 9G) ZEB1 knockdown was confirmed via western blot relative to GAPDH. (FIG. 9H) Control and shZEB1 cells were exposed to AgNPs (25 nm) for 48 h and viability was assessed via MTT assay (ANOVA; T-Test; *p<0.05, **p<0.01, ***<0.001). (FIG. 9I) Cells were incubated with the ROS responsive dye, H2DCF-DA, diluted in PBS and then ROS was assessed by fluorescence microscopy.

### EXAMPLE 10

### Ovarian Cancer Cell Lines That are Sensitive to AgNP Treatment Can Be Identified Via

ZEB1^{high}/ESRP1^{low} Biomarker Pair

A panel of ovarian cancer cells lines of were treated with AgNPs (25 nm) for (FIG. 10A) 48 h or (FIG. 10B) 72 h. Viability was assessed by MTT. (FIG. 10C) Cells were incubated with the ROS responsive dye, H2DCF-DA, diluted in PBS and then ROS was assessed by fluorescence microscopy. (FIG. 10D) mRNA expression data obtained from the Broad Institute database for ZEB1 and ESRP1 in ovarian cancer cell lines. ZEB1 expression was plotted against ESRP1 expression. (FIG. 10E) ZEB1 and ESRP1 protein expression was assessed via western blot in a panel of ovarian cancer cell lines.

### EXAMPLE 11

### The ZEB1^{hi}/ESRP1^{low} Biomarker Pair is Sufficient to Identify AgNP Sensitive Lung, Colorectal and Prostate Cancer Cell Lines

mRNA expression data obtained from the Broad Institute database for ZEB1 and ESRP1 in (FIG. 11A) lung, (FIG. 11C) colorectal and (FIG. 11E) prostate cancer cell lines was analyzed (blue: insensitive cell line tested, red: sensitive cell line tested, grey: untested cell lines). For each cancer type, ZEB1 expression was plotted against ESRP1 expression. Paired (FIG. 11B) lung, (FIG. 11D) colorectal and (FIG. 11F) prostate cancer cell lines that were ZEB1^{high}/ESRP1^{low} and ZEB1^{low}/ESRP1^{high} were treated with AgNPs (25 nm) for 72 h and viability was assessed via MTT assays. IC₅₀ doses were calculated based upon 3 independent experiments for each cell line tested.

### EXAMPLE 12

### Proposed Mechanism of Action for AgNPs in Mesenchymal Cancers Compared to Epithelial Cancers

Mesenchymal cancer cells can be identified by high ZEB1 expression and low ESRP1 expression. These cells have high basal levels of ROS. When the cells are treated with AgNPs and the AgNPs are internalized, the pro-oxidative environment induces degradation of the AgNPs into Ag⁺. The Ag⁺ then induces DNA damage, additional ROS production, and ER stress. A feedback loop occurs where as more AgNPs are internalized, more Ag⁺ is generated, and additional damage occurs until the cell undergoes apoptotic cell death. Conversely, the epithelial cells which can be identified by low ZEB1 expression and high ESRP1 expression, have low basal levels of ROS. Therefore when the epithelial-like cells are treated with AgNPs, while more AgNPs may be cell associated or internalized, only a small proportion of the AgNPs are degraded into Ag⁺, and the majority of the AgNPs are sequestered in vesicles. Therefore, while there is a slight increase in ROS, there is no significant increase in DNA damage or ER stress which is insufficient to induce apoptotic cell death.

### EXAMPLE 13

### AgNP Sensitivity is Inversely Correlated With ZEB1 Expression and Positively Correlated With ESRP1 and CDH1 Expression

Cancerous and non-tumorigenic cell lines were each assessed for their AgNP IC₅₀ values. The IC₅₀ value of each cell line was then plotted against its expression of either ZEB1 (FIG. 13), ESRP1 (FIG. 14), or CDH1 (FIG. 15). Results demonstrate that cells having greater expression of ZEB1 are more sensitive to AgNP treatment. That is, the more ZEB1 a cell line expresses, a lower the concentration of AgNPs is required to effect a response in the cell line. Conversely, cells having greater expression of ESRP1 or CDH1 were less sensitive to AgNPs. Therefore, the more ESRP1 or CDH1 a cell line expresses, a greater the concentration of AgNPs is necessary to effect a response.

### EXAMPLE 14

### HSF 1-Mediated Proteotoxic Stress Response is Inhibited by AgNP Treatment in ZEB1 Expressing Cells

BT549 claudin-low breast cancer cells and immortalized mammary epithelial cells (IMECs) were treated with increasing concentrations of AgNP and assessed by Western Blot analysis (FIG. 16) for activation of HSF1. Results demonstrate that BT549 cells exhibit dose-dependent HSF1 repression in response to silver nanoparticles. That is, activation of HSF1, via phosphorylation, is repressed by AgNP treatment in a dose-dependent manner. Furthermore, expression of HSP90, a target of HSF1, decreases with AgNP treatment in a dose-dependent manner. In contrast, activation of HSF1 and HSP90 expression in IMECs is not affected by

AgNP treatment.

### EXAMPLE 16

### AgNP Treatment Synergizes With HSF1 Inhibition in Breast Cancer Cells

BT549 claudin-low breast cancer cells were treated with either AgNPs or KRIBB11, an HSF1 inhibitor, independently and in combination. The treated cells were then assessed for cell viability by MTT assay (FIG. 17A). Results demonstrate BT549 cells are equally sensitive to either AgNPs or HSF1 inhibition (FIG. 17B). However, combination treatment of both AgNPs and KRIBB11 resulted in a synergistic response (FIG. 17C).

BT20 basal-like breast cancer cells were treated with either AgNPs or KRIBB11, an HSF1 inhibitor, independently and in combination. Treated cells were then assessed for cell viability by MTT assay (FIG. 18A). Results demonstrate BT20 cells are marginally more sensitive to AgNPs than HSF1 inhibition (FIG. 18B). However, combination treatment of both AgNPs and KRIBB11 resulted in synergistic response (FIG. 18C).

### EXAMPLE 17

### AgNP Treatment Compliments HSP90 Inhibition in Breast Cancer Cells

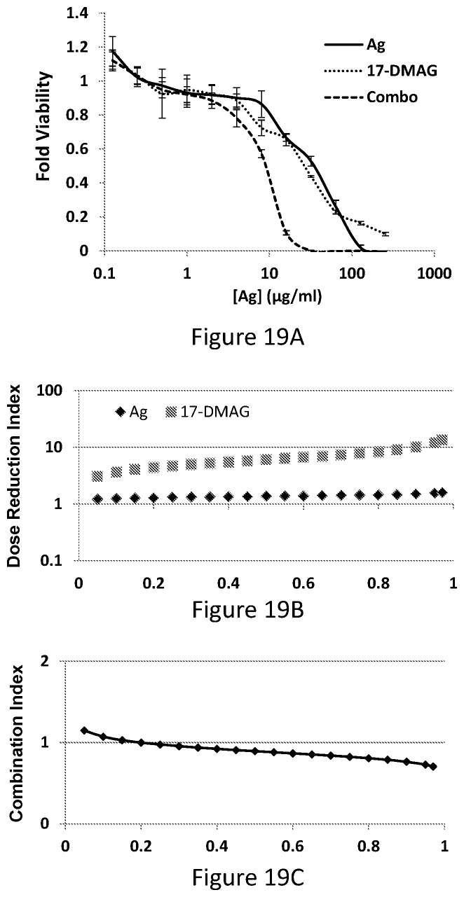

BT549 claudin-low breast cancer cells were treated with either AgNPs or 17-DMAG, an HSP90 inhibitor, independently and in combination. Treated cells were then assessed for cell viability by MTT assay (FIG. 19A). Results demonstrate BT549 cells are equally sensitive to either AgNPs or HSP90 inhibition (FIG. 19B). Combination treatment of both AgNPs and

KRIBB11 resulted in an additive response (FIG. 19C).

1. A method of treating cancer comprising:
quantifying ZEB1 expression in a cancerous cell population;
comparing the quantified ZEB1 expression with a ZEB1 expression threshold above which the cancerous cell population responds to silver nanoparticles; and
administering silver nanoparticles to the cancerous cell population if the quantified ZEB1 expression meets or exceeds the ZEB1 expression threshold.

2. The method of claim 1, wherein the cancerous cell population comprises triple-negative breast cancer cells.

3. The method of claim 2, wherein the triple-negative breast cancer cells comprise claudin-low subtypes.

4. The method of claim 1, wherein the cancerous cell population comprises at least one of lung cancer cells, colorectal cancer cells, ovarian cancer cells and prostate cancer cells.

5. The method of claim 1, wherein the silver nanoparticles are administered at a concentration of silver of 1 μg/ml to 100 mg/ml.

6. The method of claim 1, wherein the silver nanoparticles are administered at a concentration of silver of 5 μg/ml to 50 μg/ml.

7. The method of claim 1, wherein the silver nanoparticles have an average size of 5 nm to 50 nm.

8. The method of claim 1, wherein the silver nanoparticles have an average size of 5 nm to 30 nm.

9. The method of claim 1, wherein the silver nanoparticles comprise a polymeric coating.

10. The method of claim 1, wherein the silver nanoparticles comprise a silica coating.

11. The method of claim 1, wherein the silver nanoparticles are in the ground state.

12. The method of claim 1 further comprising quantifying ESRP1 and/or CDH1 expression in the cancerous cell population.

13. The method of claim 11, wherein ESRP1 and/or CDH1 expression is less than ZEB1 expression.

14. The method of claim 1 wherein the silver nanoparticles selectively kill the cancerous cell population.

15. The method of claim 1, wherein the silver nanoparticles are administered intravenously.

16. The method of claim 1, wherein the cancerous cell population comprises claudin-low subtypes.

17. The method of claim 1, wherein the cancerous cell population comprises elevated reactive oxygen species that trigger pH-dependent ionization of the silver nanoparticles.

18. A method of determining response of a cancerous cell population to silver nanoparticles comprising:
quantifying ZEB1 expression in the cancerous cell population; and
comparing the quantified ZEB1 expression with a ZEB1 expression threshold above which the cancerous cell population responds to the silver nanoparticles.

19. The method of claim 18 further comprising:
quantifying ESRP1 expression in the cancerous cell population; and
comparing the quantified ESRP1 expression with an ESRP1 expression threshold below which the cancerous cell population responds to the silver nanoparticles.

20. The method of claim 18 further comprising:
quantifying CDH1 expression in the cancerous cell population; and
comparing the quantified CDH1 expression with an CDH1 expression threshold below which the cancerous cell population responds to the silver nanoparticles.

21. The method of claim 18 further comprising:
quantifying HSF1 activation in the cancerous cell population; and
comparing the quantified HSF1 activation with an HSF1 activation threshold below which the cancerous cell population responds to the silver nanoparticles.

22. The method of claim 1, wherein the silver nanoparticles inhibit activation of HSF1.

23. The method of claim 1, further comprising administering one or more heat shock inhibitors to the cancerous cell population.

24. The method of claim 23, wherein the one or more heat shock inhibitors inhibits HSF1 and/or one or more heat shock proteins.

25. The method of claim 23 wherein the one or more heat shock inhibitors synergize with the silver nanoparticles.

26. The method of claim 1 further comprising:
quantifying HSF1 activation;
comparing the quantified HSF1 activation with an HSF1 activation threshold above which the cancerous cell population responds to an HSF1 inhibitor; and
administering an HSF1 inhibitor to the cancerous cell population if the quantified HSF1 activation meets or exceeds the HSF1 activation threshold.
